(19) United States
(12) Patent Application Publication
(10) Pub. No.: US 2026/0325568 A1
(43) Pub. Date: Oct. 22, 2026

(54) Formulations and Methods for Treating Symptoms of Menopause

(71) Applicant: Kindra Corporation, Santa Monica, CA (US)

(72) Inventors: Hasti Fay Nazemzadeh, San Francisco, CA (US); Sundeep Singh Gill, Hayward, CA (US)

(73) Assignee: Kindra Corporation, Santa Monica, CA (US)

(21) Appl. No.: 19/728,559

(22) Filed: Jul. 2, 2026

Related U.S. Application Data
(63) Continuation of application No. 18/710,370, filed on Nov. 18, 2022.
(60) Provisional application No. 63/281,175, filed on Nov. 19, 2021.
(60) Provisional application No. 63/414,674, filed on Oct. 10, 2022.

(51) Int. Cl. A61K 31/728; A61K 9/00; A61K 31/198; A61K 31/205; A61K 31/355; A61K 31/455; A61K 36/889; A61K 38/08; A61K 45/06; A61K 47/10; A61K 47/12; A61K 47/24; A61K 47/36; A61K 47/44; A61P 15/00

(52) U.S. Cl. CPC A61K 31/728; A61K 9/0014; A61K 31/198; A61K 31/205; A61K 31/355; A61K 31/455; A61K 36/889; A61K 38/08; A61K 45/06; A61K 47/10; A61K 47/12; A61K 47/24; A61K 47/36; A61K 47/44; A61P 15/00

(57) ABSTRACT

The present disclosure relates to aqueous topical formulations comprising at least one TRPV1 antagonist and at least one specific plant extract. Also disclosed herein are methods of treating at least one symptom of menopause comprising topically administering an effective amount of the formulations disclosed.

### FIELD

Disclosed herein are aqueous topical formulations comprising at least one TRPV1 antagonist and at least one plant extract, as well as methods of treating menopause-related symptoms with the formulations disclosed herein.

### BACKGROUND and SUMMARY OF DISCLOSURE

Perimenopause and menopause can result from a variety of events, including naturally declining reproductive hormones, surgery that removes the ovaries, chemotherapy, radiation therapy, lactation, hypothalamic dysfunction, and primary ovarian insufficiency. Regardless of the onset event, women undergoing perimenopause and menopause have diminishing estrogen levels that can lead to a collection of physical symptoms including hot flashes, night sweats, and a syndrome recently referred to as Genitourinary Syndrome of Menopause (GSM). Women experiencing GSM may present with a variety of issues related to physical changes of the vulva, vagina, and lower urinary tract, including, vaginal dryness and irritation; burning sensations; dyspareunia; pain; throbbing; itching; stinging; frequent yeast infections; feeling of pressure; yellow malodorous discharge; tenderness; urinary frequency, incontinence, and urgency; urinary tract infections (UTIs); difficulty in sexual arousal; inadequate lubrication during arousal; vaginal bleeding from fragile atrophic skin; and dryness of labia. Wańczyk-Baszak et al., Menopause Rev 2018; 17(4): 180-184.

Vulvovaginal atrophy (VVA) and vulvodynia (VVD) have symptoms that can be components of GSM, or that may result from separate, individual diagnoses. Vulvovaginal atrophy is characterized by thinning, dryness, inflammation of the vaginal walls, decreased lubrication, and compromised skin barrier function. Vulvodynia is characterized by painful burning sensation, stinging, rawness, soreness, throbbing, allodynia, and hyperalgesia in the region of the vulval vestibulus. Due to overlapping symptoms (e.g, inflammation, dyspareunia, burning, vulvar itching), misdiagnosis of one for the other is understandable and common. Mitro et al., Women's Midlife Health (2016) 2:4. However, misdiagnosis and treatment of VVA when the subject actually has VVD results not only in a failure to treat the true source of discomfort and reduce symptoms, but may additionally lead to worsening of symptoms. For example, formulations designed for VVA may not be stable in the pH of a vagina that has VVD. Thus, exposure to formulations that may contain ingredients that are unstable in the environment where they are applied can lead to further irritation and/or exacerbation of symptoms instead of relief.

Further complicating diagnoses based on symptoms is the role estrogen plays in supporting regulation of healthy vaginal pH in women. As noted already herein, women undergoing perimenopause and menopause have diminishing estrogen levels. Thus, as estrogen declines through perimenopause and menopause, vaginal pH shifts from acidic to alkaline. Endrocinology (2005) February; 146(2): 816-824. Given the variation of vaginal pH throughout the menopause experience, pH is an important consideration in topical formulations. Certain ingredients that may be standard in vulvar and vaginal formulations, such as niacinamide, may decompose in an environment with pH higher or lower than 6. Thus decomposition of ingredients in formulations intended to bring relief or minimize discomfort, may instead cause irritation. And the application of currently existing topical formulations to already hypersensitive vulvovaginal tissue in people experiencing VVD or VVA has been shown to exacerbate existing irritation caused by these conditions.

Observations from rodent models and human patients appear to show a link between hormone levels and the establishment of vaginal hypersensitivity, and that altered sensitivity involves the proliferation of sensory nerves within vaginal tissues. Ting et al., Bio. Of Reproduction, 71, 1397-1404 (2004). Moreover, one study found subjects suffering from vulvodynia have, among other things, increased expression of transient receptor potential vanilloid type 1 (TRPV1) receptors, indicating increased activity. Tympanidis et al., European J. of Pain 8 (2004) 129-133. The TRPV1 receptor is expressed by nociceptor fibers and is triggered by capsaicin, noxious heat, protons, and chemicals produced during inflammation. Id. The TRPV1 receptor is an ion channel involved in transmitting and modulating thermal pain sensation from sensory neurons to the brain. Thus, treatment for a subject suffering from symptoms that could be vulvodynia may benefit from more specific targeting of pain receptors including TRVP1, for example if reduced estrogen leads to further activation of TRPV1 in the vaginal tissues.

Accordingly, the present disclosure relates to aqueous topical formulations comprising at least one TRPV1 antagonist; and at least one plant extract chosen from Chamomilla recutita flower extract, Sapindus trifoliatus fruit extract, aloe leaf juice, cucumber extract, hydrolyzed quinoa, Hamamelis virginiana leaf extract, oat kernel extract, oat kernel flour, coconut extract, Rosa damascena flower extract, microalgae extract, Akebia quinata extract, Rhodosorus marinus extract, Phaeodactylum tricornutum extract, conifer extract, Blumea balsamifera extract, Kaempferia galanga extract, coriander extract, Coriandrum sativum extract, Citrus sinensis extract, lavender extract, Lavandula angustifolia extract, bay laurel extract, sweet basil extract, Ocimum basilicum extract, Ocimum tenuiflorum extract, mint extract, Mentha piperita extract, Mentha spicata extract, Mentha haplocalyx extract, citronella oil extract, rose oil extract, palmarosa oil extract, geranium extract, Cymbopogon citratus extract, Corymbia citriodora extract, Prunus mandschurica extract, apple tree leaf extract, cinnamon bark extract, strawberry extract, Angelica sinensis extract, acai oil extract, Mangifera indica extract, Fumaria officinalis extract, Rumex japonicus extract, guaiacwood extract, Guaiaicum officinale extract, Guaiacum sanctum extract, lemongrass extract, citrus oil extract, Citrus bergamia extract, Vanilla planifolia extract, Vanilla tahitensis extract, Vanilla pompona extract, Theobroma cacao extract, pomegranate extract, Myrciaria dubia extract, Houttuynia cordata extract, sea lettuce extract, Ulva compressa extract, Agathosma betulina extract, Mentha canadensis extract, Eclipta prostrate extract, calendula extract, Silybum marianum extract, Cydonia oblonga extract, Oenothera biennis extract, Lepidium meyenii extract, Ulmus rubra extract, Olea europaea extract, Acmella oleracea extract, Humulus lupulus extract, Nicotiana sylvestris extract, jasmine extract, Cananga odorata extract, quince extract, olive tree extract, Vaccinium oxycoccus extract, Vaccinium macrocarpon extract, prickly pear cactus extract, Gymnema sylvestre extract, purslane extract, Rosa penduline extract, Ceratonia siliqua extract, Prunus amygdalus extract, Prunus dulcis extract, Prunus armeniaca extract, Ginkgo biloba extract, avocado extract, Persea americana extract, Camellia sinensis extract, eucalyptus extract, Linum usitatissimum extract, Cyamopsis tetragonoloba extract, sea buckthorn extract, Helianthus annuus extract, Simmondsia chinensis extract, Limnanthes alba extract, Sesamum indicum extract, Azadirachta indica extract, Moringa oleifera extract, Cedrus atlantica extract, calophyllum extract, Calophyllum inophyllum extract, Calophyllum tacamahaca extract, Daucus carota extract, Daucus carota sativa extract, Euphorbia cerifera extract, candelilla extract, carnauba palm extract, Echinacea purpurea extract, Zea mays extract, Quercus infectoria extract, arrowroot extract, Eurocoma longifolia extract, Trigonella foenum-graecum extract, salvia extract, rosemary extract, sage extract, clary sage extract, Oryza sativa extract, Zingiber officinale extract, licorice extract, Matricaria recutita extract, oil palm extract, tapioca extract, Vitis vinifera extract, hazelnut tree extract, Turnera diffusa extract, violet extract, Podocarpus totara extract, raspberry extract, Rubus idaeus extract, Rubus strigosus extract, wild indigo extract, safflower extract, and apple extract.

The present disclosure also relates to a method of treating at least one symptom of menopause in a subject suffering therefrom, comprising topically administering to the subject an effective amount of an aqueous topical formulation comprising at least one TRPV1 antagonist; and at least one plant extract chosen from Chamomilla recutita flower extract, Sapindus trifoliatus fruit extract, aloe leaf juice, cucumber extract, hydrolyzed quinoa, Hamamelis virginiana leaf extract, oat kernel extract, oat kernel flour, coconut extract, Rosa damascena flower extract, microalgae extract, Akebia quinata extract, Rhodosorus marinus extract, Phaeodactylum tricornutum extract, conifer extract, Blumea balsamifera extract, Kaempferia galanga extract, coriander extract, Coriandrum sativum extract, Citrus sinensis extract, lavender extract, Lavandula angustifolia extract, bay laurel extract, sweet basil extract, Ocimum basilicum extract, Ocimum tenuiflorum extract, mint extract, Mentha piperita extract, Mentha spicata extract, Mentha haplocalyx extract, citronella oil extract, rose oil extract, palmarosa oil extract, geranium extract, Cymbopogon citratus extract, Corymbia citriodora extract, Prunus mandschurica extract, apple tree leaf extract, cinnamon bark extract, strawberry extract, Angelica sinensis extract, acai oil extract, Mangifera indica extract, Fumaria officinalis extract, Rumex japonicus extract, guaiacwood extract, Guaiaicum officinale extract, Guaiacum sanctum extract, lemongrass extract, citrus oil extract, Citrus bergamia extract, Vanilla planifolia extract, Vanilla tahitensis extract, Vanilla pompona extract, Theobroma cacao extract, pomegranate extract, Myrciaria dubia extract, Houttuynia cordata extract, sea lettuce extract, Ulva compressa extract, Agathosma betulina extract, Mentha canadensis extract, Eclipta prostrate extract, calendula extract, Silybum marianum extract, Cydonia oblonga extract, Oenothera biennis extract, Lepidium meyenii extract, Ulmus rubra extract, Olea europaea extract, Acmella oleracea extract, Humulus lupulus extract, Nicotiana sylvestris extract, jasmine extract, Cananga odorata extract, quince extract, olive tree extract, Vaccinium oxycoccus extract, Vaccinium macrocarpon extract, prickly pear cactus extract, Gymnema sylvestre extract, purslane extract, Rosa penduline extract, Ceratonia siliqua extract, Prunus amygdalus extract, Prunus dulcis extract, Prunus armeniaca extract, Ginkgo biloba extract, avocado extract, Persea americana extract, Camellia sinensis extract, eucalyptus extract, Linum usitatissimum extract, Cyamopsis tetragonoloba extract, sea buckthorn extract, Helianthus annuus extract, Simmondsia chinensis extract, Limnanthes alba extract, Sesamum indicum extract, Azadirachta indica extract, Moringa oleifera extract, Cedrus atlantica extract, calophyllum extract, Calophyllum inophyllum extract, Calophyllum tacamahaca extract, Daucus carota extract, Daucus carota sativa extract, Euphorbia cerifera extract, candelilla extract, carnauba palm extract, Echinacea purpurea extract, Zea mays extract, Quercus infectoria extract, arrowroot extract, Eurocoma longifolia extract, Trigonella foenum-graecum extract, salvia extract, rosemary extract, sage extract, clary sage extract, Oryza sativa extract, Zingiber officinale extract, licorice extract, Matricaria recutita extract, oil palm extract, tapioca extract, Vitis vinifera extract, hazelnut tree extract, Turnera diffusa extract, violet extract, Podocarpus totara extract, raspberry extract, Rubus idaeus extract, Rubus strigosus extract, wild indigo extract, safflower extract, and apple extract.

The present disclosure still further relates to the use of any of the aqueous topical formulations disclosed herein to help treat and/or ameliorate at least one symptom of menopause.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

### DETAILED DESCRIPTION

Reference will now be made in detail to certain embodiments. While the disclosure provides illustrated embodiments, it will be understood that they are not intended to limit the disclosure to those embodiments. On the contrary, the disclosure is intended to cover all alternatives, modifications, and equivalents, which may be included within the disclosure as defined by the appended claims.

Any section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

A subject can experience a reduction of their normal estrogen level for a variety of reasons, including due to natural physiological circumstances, such as experiencing premenopause, perimenopause, menopause, postmenopause, or lactation; due to induced medical or surgical intervention, such as undergoing chemotherapy, radiation, an oophorectomy or ovariotomy, a hysterectomy, or by taking selective estrogen receptor modulators, selective estrogen receptor degraders, or antigonadotropins; or due to another condition such as a hypothalamic dysfunction, pregnancy failure, anorexia, or polycystic ovarian syndrome.

The compositions and methods described herein may be applicable to treating, alleviating, and/or modulating at least one symptom in a subject experiencing a reduced estrogen level for at least one of the reasons described above. In at least one embodiment, the reduction in estrogen level is a result of a subject experiencing perimenopause or menopause. In at least one embodiment, the reduction in estrogen level is a result of a subject experiencing menopause. In at least one embodiment, the reduction in estrogen level is a result of a subject undergoing chemotherapy and/or radiation. In at least one embodiment, the reduction in estrogen level is a result of a subject undergoing an oophorectomy or ovariotomy. In at least one embodiment, the reduction in estrogen level is a result of a subject undergoing a hysterectomy. In at least one embodiment, the reduction in estrogen level is a result of a subject undergoing antigonadotropin therapy. In at least one embodiment, the reduction in estrogen level is a result of a subject undergoing selective estrogen receptor modulator therapy.

As used herein, the term estrogen refers to estrogens having hormonal activity, including estrone, estradiol, and estriol. In at least one embodiment, the term estrogen refers to estradiol. In at least one embodiment, the term estrogen refers to at least one of estrone, estradiol, and estriol. In at least one embodiment, the term estrogen refers to at least one of estrone, estradiol, and estriol. In at least one embodiment, the term estrogen comprises at least one of estrone, estradiol, and estriol. In at least one embodiment, the term estrogen comprises estradiol.

Normal blood estrogen levels range from about 30 to about 400 pg/mL for premenopausal women and from about zero to about 30 pg/mL for postmenopausal women. In a study of postmenopausal subjects not using hormone therapy and experiencing chronic vulvar pain, for example, blood serum hormone levels of estradiol ranged from 12.0 to 27.2 pg/mL, with an average of 19.8 pg/mL. Mitro et al., Women's Midlife Health, 2:4, (2016).

In at least one embodiment, a subject experiencing a reduced, or lower than normal, level of estrogen has a blood estrogen level ranging from about zero to about 300 pg/mL, such as ranging from about zero to about 200 pg/mL, from about 5 to about 100 pg/mL, from about 10 to about 50 pg/mL, or that is about 10 pg/mL, about 15 pg/mL, about 20 pg/mL, about 25 pg/mL, about 30 pg/mL, or about 50 pg/mL. In at least one embodiment, a subject experiencing a reduced level of estrogen has a blood estrogen level of not greater than about 5 pg/mL, not greater than about 10 pg/mL, not greater than about 15 pg/mL not greater than about 20 pg/mL, not greater than about 30 pg/mL, or not greater than about 50 pg/mL. In at least one embodiment, a subject experiencing a reduced level of estrogen has a blood estrogen level that is not measurable, or that is about zero.

A subject experiencing a reduced level of estrogen may have a level of estrogen that ranges from about 10% to about 99% of a normal level, such as from about 50% to about 99% of a normal level, from about 60% and about 95% of a normal level, or from about 75% to about 90% of a normal level. In at least one embodiment, a subject experiencing a reduced level of estrogen may have a level of estrogen that is not greater than about 50% of a normal level, not greater than about 75% of a normal level, or not greater than about 95% of a normal level. In at least one embodiment, a subject experiencing a reduced level of estrogen may have a level of estrogen that is about 5%, about 10%, about 15%, about 20%, about 25%, about 30%, about 35%, about 40%, about 50%, about 60%, or about 75% of a normal level. In at least one embodiment, a subject experiencing a reduced level of estrogen may have a level of estrogen that is about 20% of normal, about 30% of normal, about 40% of normal, about 50% of normal, about 60% of normal, about 70% of normal, about 80% of normal, about 90% of normal, or about 95% of normal.

The compositions and methods described herein may be applicable to treating, alleviating, and/or moderating at least one symptom of a reduced estrogen level. A subject having a reduced level of estrogen, also referred to as being hypoestrogenic, can experience at least one symptom, including experiencing multiple symptoms. Symptoms of a hypoestrogenic state include vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, dysuria, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, allodynia, and hyperalgesia in the region of the vulval vestibulus.

A subject can experience symptoms that may be related to reduced estrogen levels due to a natural condition, such as premenopause, perimenopause, menopause, postmenopause, or lactation. Menopause-related symptoms include, for example, hot flashes, night sweats, dry skin as related to menopausal transition, irregular menstruation, mood changes, difficulty sleeping, and osteoporosis. A subject can experience symptoms that may be related to reduced estrogen levels due to medical or surgical intervention, such as undergoing chemotherapy, radiation, an oophorectomy, an ovariotomy, a hysterectomy, or by taking selective estrogen receptor modulators, selective estrogen receptor degraders, or antigonadotropins. A subject can experience symptoms that may be related to reduced estrogen levels due to a disease or disorder, such as a hypothalamic dysfunction, pregnancy failure, anorexia, polycystic ovarian syndrome, VVD, VVA, GSM, atrophic vaginitis, or vestibulodynia. A subject can experience symptoms that may be related to reduced estrogen levels due to at least one condition, or from multiple conditions, including any combination of the conditions described herein.

The compositions and methods described herein may be applicable to treating, alleviating, and/or moderating at least one menopause-related symptom. A menopause-related symptom may result from temporarily or permanently reduced estrogen levels in a subject. A temporarily reduced estrogen level may, for example, occur from a subject lactating or undergoing chemotherapy, whereas a permanently reduced estrogen level may, for example, occur after a hysterectomy or during postmenopause. A menopause-related symptom includes vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

A hypoestrogenic condition can manifest as an imbalance of the vulvovaginal flora that results in an altered pH. A constituent of vaginal microbiota is lactobacillus, which produce lactic acid and maintains the optimum low pH of the vaginal fluid. Naumova et al., Int. J. of Women's Health, 10, 387-395 (2018). The low pH protects from infections of the urogenital tract, and under conditions of estrogen deficiency, the balance of microbiota is disrupted and the vagina can develop a less acidic pH, such as from 5.5 to 6.8. Id. The shift in normal flora can result a decrease in Lactobacillus ssp., which can result in overgrowth of skin and rectal pathogens, such as, for example, the pathogenic yeast Candida albicans. Flores, et al., StatPearls (2020), StatPearls Publishing: https://www.ncbi.nlm.nih.gov/books/NBK564341/; retrieved Aug. 31, 2021; De Andres, et al., Pain Practice, 16:2, 204-236 (2016).

Disclosed herein are aqueous topical formulations comprising at least one TRPV1 antagonist and at least one plant extract chosen from Chamomilla recutita flower extract, Sapindus trifoliatus fruit extract, aloe leaf juice, cucumber extract, hydrolyzed quinoa, Hamamelis virginiana leaf extract, oat kernel extract, oat kernel flour, coconut extract, Rosa damascena flower extract, microalgae extract, Akebia quinata extract, Rhodosorus marinus extract, Phaeodactylum tricornutum extract, conifer extract, Blumea balsamifera extract, Kaempferia galanga extract, coriander extract, Coriandrum sativum extract, Citrus sinensis extract, lavender extract, Lavandula angustifolia extract, bay laurel extract, sweet basil extract, Ocimum basilicum extract, Ocimum tenuiflorum extract, mint extract, Mentha piperita extract, Mentha spicata extract, Mentha haplocalyx extract, citronella oil extract, rose oil extract, palmarosa oil extract, geranium extract, Cymbopogon citratus extract, Vorymbia citriodora extract, Prunus mandschurica extract, apple tree leaf extract, cinnamon bark extract, strawberry extract, Angelica sinensis extract, acai oil extract, Mangifera indica extract, Fumaria officinalis extract, Rumex japonicus extract, guaiacwood extract, Guaiaicum officinale extract, Guaiacum sanctum extract, lemongrass extract, citrus oil extract, Citrus bergamia extract, Vanilla planifolia extract, Vanilla tahitensis extract, Vanilla pompona extract, Theobroma cacao extract, pomegranate extract, Myrciaria dubia extract, Houttuynia cordata extract, sea lettuce extract, Ulva compressa extract, Agathosma betulina extract, Mentha canadensis extract, Eclipta prostrate extract, calendula extract, Silybum marianum extract, Cydonia oblonga extract, Oenothera biennis extract, Lepidium meyenii extract, Ulmus rubra extract, Olea europaea extract, Acmella oleracea extract, Humulus lupulus extract, Nicotiana sylvestris extract, jasmine extract, Cananga odorata extract, quince extract, olive tree extract, Vaccinium oxycoccus extract, Vaccinium macrocarpon extract, prickly pear cactus extract, Gymnema sylvestre extract, purslane extract, Rosa penduline extract, Ceratonia siliqua extract, Prunus amygdalus extract, Prunus dulcis extract, Prunus armeniaca extract, Ginkgo biloba extract, avocado extract, Persea americana extract, Camellia sinensis extract, eucalyptus extract, Linum usitatissimum extract, Cyamopsis tetragonoloba extract, sea buckthorn extract, Helianthus annuus extract, Simmondsia chinensis extract, Limnanthes alba extract, Sesamum indicum extract, Azadirachta indica extract, Moringa oleifera extract, Cedrus atlantica extract, calophyllum extract, Calophyllum inophyllum extract, Calophyllum tacamahaca extract, Daucus carota extract, Daucus carota sativa extract, Euphorbia cerifera extract, candelilla extract, carnauba palm extract, Echinacea purpurea extract, Zea mays extract, Quercus infectoria extract, arrowroot extract, Eurocoma longifolia extract, Trigonella foenum-graecum extract, salvia extract, rosemary extract, sage extract, clary sage extract, Oryza sativa extract, Zingiber officinale extract, licorice extract, Matricaria recutita extract, oil palm extract, tapioca extract, Vitis vinifera extract, hazelnut tree extract, Turnera diffusa extract, violet extract, Podocarpus totara extract, raspberry extract, Rubus idaeus extract, Rubus strigosus extract, wild indigo extract, safflower extract, and apple extract.

In some embodiments, the at least one TRPV1 antagonist is present in an amount ranging from 0.0001% w/w to 7% w/w, such as from 0.001% w/w to 5% w/w, from 0.01% w/w to 3.5% w/w, and from 0.1% w/w to 3.0% w/w. In some embodiments, the total amount of plant extract is present in an amount ranging from 0.00001% w/w to 4.0% w/w, such as from 0.0001% w/w to 3.5% w/w, from 0.001% w/w to 2.5% w/w, from 0.01% w/w to 1.5%, and from 0.1% to 1% w/w. In at least one embodiment, the at least one TRPV1 antagonist is present in an amount ranging from 0.001% w/w to 4% w/w, and the total amount of plant extract is present in an amount ranging from 0.001% w/w to 4% w/w.

The aqueous topical formulations disclosed herein can further comprise sodium hyaluronate and/or other hyaluronic acid salts and/or derivatives. In some embodiments, the sodium hyaluronate is present in an amount ranging from 0.0001% w/w to 5% w/w, such as from 0.001% w/w to 4% w/w, from 0.01% w/w to 2.5% w/w, and from 0.1% w/w to 1% w/w. In at least one embodiment, the aqueous topical formulation comprises sodium hyaluronate in an amount ranging from 0.001% w/w to 1% w/w.

The aqueous topical formulations disclosed herein can further comprise at least one form of vitamin E. In some embodiments, the vitamin E is present in an amount ranging from 0.0000001% w/w to 2.0% w/w, such as from 0.000001% w/w to 1% w/w, and from 0.00001% w/w to 0.1% w/w. In at least one embodiment, the aqueous topical formulation comprises vitamin E in an amount ranging from 0.0000001% w/w to 1% w/w.

The aqueous topical formulations disclosed herein can further comprise coconut oil. In some embodiments, the coconut oil is present in an amount ranging from 0.0000001% w/w to 1.0% w/w, such as from 0.000001% w/w to 0.5% w/w, from 0.00001% w/w to 0.2%, and from 0.0001% to 0.1% w/w. In at least one embodiment, the aqueous topical formulation comprises coconut oil in an amount ranging from 0.0001% w/w to 1% w/w.

The aqueous topical formulations disclosed herein can further comprise betaine. In some embodiments, the betaine is present in an amount ranging from 0.00001% w/w to 3.0% w/w, such as from 0.0001% w/w to 2.0%, from 0.001% w/w to 1.0% w/w, and from 0.01% w/w to 0.5% w/w. In at least one embodiment, the aqueous topical formulation comprises betaine in an amount ranging from 0.001% w/w to 1% w/w.

The aqueous topical formulations disclosed herein can further comprise arginine. In some embodiments, the arginine is present in an amount ranging from 0.00001% w/w to 3.0% w/w, such as from 0.0001% w/w to 2.0%, from 0.001% w/w to 1.0% w/w, and from 0.01% w/w to 0.5% w/w. In at least one embodiment, the aqueous topical formulation comprises arginine in an amount ranging from 0.01% w/w to 1% w/w.

The aqueous topical formulations disclosed herein can further comprise niacinamide. In some embodiments, the niacinamide is present in an amount less than 3.0% w/w, such as less than 2.0% w/w, less than 1.0% w/w, less than 0.5% w/w, less than 0.1% w/w, less than 0.01% w/w, or less than 0.0001% w/w. In at least one embodiment, the aqueous topical formulation does not comprise niacinamide. In at least one embodiment, the aqueous topical formulation is substantially free of niacinamide.

The aqueous topical formulations disclosed herein can be made without including, or made free of, certain ingredients. For example, ingredients that are hormonally active and/or disrupt endocrine systems, such as parabens, phthalates, or xenoestrogens, may be undesirable for use in a formulation suitable for hypoestrogenic subjects. Similarly, cancer-causing ingredients or ingredients suspected of being carcinogenic, such as formaldehyde, may be undesirable, as are ingredients that may cause an allergic reaction, such as gluten, soy, nut, mineral oil, or fragrance. In at least one embodiment, the aqueous topical formulation does not comprise at least one of niacinamide, estrogen, progesterone, paraben, phthalate, sulfate, gluten, fragrance, soy, nut, mineral oil, and formaldehyde. In at least one embodiment, the aqueous topical formulation is substantially free of at least one of niacinamide, estrogen, progesterone, paraben, phthalate, sulfate, gluten, fragrance, soy, nut, mineral oil, and formaldehyde.

Similarly, the aqueous topical formulations disclosed herein can be made without including, or made free of, ingredients that may irritate the vulvovaginal environment. Irritating ingredients that may shift the pH or osmolality of the formulation toward values outside of normal, such as citric acid in concentrations that render the formulation very acidic (that is, below a pH of about 3.5), may be undesired. In at least one embodiment, the aqueous topical formulation does not comprise citric acid.

As used herein, the term “substantially free” of an ingredient refers to containing less than about 1% by weight, such as less than about 0.5% by weight, such as less than about 0.25% by weight, and such as less than about 0.1% by weight of such ingredient. In at least one embodiment, “substantially free” means completely free of such ingredient.

The aqueous topical formulations disclosed herein can further comprise at least one pH adjuster. In at least one embodiment, the aqueous topical formulations comprise a pH adjuster. In at least one embodiment, the pH adjuster comprises lactic acid.

The aqueous topical formulations disclosed herein can further comprise at least one TRPV1 antagonist.

Non-limiting examples of TRPV1 antagonists include those chosen from Table A:

TABLE A
TRPV1 antagonists

| JYL-1421 | Brito, R., et al. (2014). Cells, 3(2), 517-545. |
|---|---|
| [N-(4-tert-butylbenzyl)-N′-[3- | Wang, Y., et al. (2002). Molecular pharmacology, |
| fluoro-4- | 62(4), 947-956. |
| (methylsulfonylamino)benzyl] | |
| thiourea] | |
| KJM429 | Brito, R., et al. (2014). Cells, 3(2), 517-545. |
| [N-(4-tert-butylbenzyl)-N′- | Wang, Y., et al. (2002). Molecular pharmacology, |
| [4-(methylsulfonylamino) | 62(4), 947-956. |
| benzyl]thiourea] | |
| A-425619 | Brito, R., et al. (2014). Cells, 3(2), 517-545. |
| [1-isoquinolin-5-yl-3-(4- | El Kouhen, R., et al. (2005). The Journal of |
| trifluoromethyl-benzyl)-urea] | pharmacology and experimental therapeutics, |
| | 314(1), 400-409. |
| BCTC | Brito, R., et al. (2014). Cells, 3(2), 517-545. |
| [N-(4-tertiarybutylphenyl)-4- | Valenzano, K. J., et al. (2003). The Journal of |
| (3-chloropyridin-2- | pharmacology and experimental therapeutics, |
| yl)tetrahydropyrazine-1(2H)- | 306(1), 377-386. |
| carbox-amide] | |
| JNJ-17203212 | Brito, R., et al. (2014). Cells, 3(2), 517-545. |
| [4-(3-trifluoromethylpyridin- | Swanson, D. M., et al. (2005). Journal of medicinal |
| 2-yl)piperazine-1-carboxylic | chemistry, 48(6), 1857-1872. |
| acid (5- | |
| trifluoromethylpyridin-2- | |
| yl)amide] | |
| SB-705498 | Brito, R., et al. (2014). Cells, 3(2), 517-545. |
| [N-(2-bromophenyl)-N′-[((R)- | Rami, H. K., et al. (2006). Bioorganic & medicinal |
| 1-(5-trifluoromethyl-2- | chemistry letters, 16(12), 3287-3291. |
| pyridyl)pyrrolidin-3-yl)]urea] | |
| SB-366791 | Brito, R., et al. (2014). Cells, 3(2), 517-545. |
| [4′-Chloro-3- | Gunthorpe, M. J., et al. (2004). |
| methoxycinnamanilide] | Neuropharmacology, 46(1), 133-149. |
| AMG-9810 | Brito, R., et al. (2014). Cells, 3(2), 517-545. |
| [(E)-3-(4-t-butylphenyl)-N- | Gavva, N. R., et al. (2005). The Journal of |
| (2,3-dihydrobenzo[b][1,4] | pharmacology and experimental therapeutics, |
| dioxin-6-yl)acrylamide] | 313(1), 474-484. |
| AMG-2674 | Brito, R., et al. (2014). Cells, 3(2), 517-545. |
| [3-Amino-5-[[2-[(2- | Ognyanov, V. I., et al. (2006). Journal of medicinal |
| methoxyethyl)amino]-6-[4- | chemistry, 49(12), 3719-3742. |
| (trifluoromethyl)phenyl]-4- | |
| pyrimidinyl]oxy]-2(1H)- | |
| quinoxalinone] | |
| Capsazepine | Brito, R., et al. (2014). Cells, 3(2), 517-545. |
| | Walpole, C. S., et al. (1994). Journal of medicinal |
| | chemistry, 37(13), 1942-1954. |
| | Yang, M. H., et al. (2019). Molecules (Basel, |
| | Switzerland), 24(5), 995. |
| MK-2295 | Brito, R., et al. (2014). Cells, 3(2), 517-545. |
| [6-((R)-4-(6-(4-fluorophenyl)- | Schaffler, K., et al. (2013). British journal of |
| 2-((R)-2-methylpyrrolidin-1- | clinical pharmacology, 75(2), 404-414. |
| yl)pyrimidin-4-yl)-3- | Kym, P. R., et al. (2009). Biochemical |
| methylpiperazin-1-yl)-5- | pharmacology, 78(3), 211-216. |
| methylnicotinic acid] | |
| Ruthenium red | Brito, R., et al. (2014). Cells, 3(2), 517-545. |
| | Garcia-Martínez, C., et al. (2000). The Journal of |
| | biological chemistry, 275(42), 32552-32558. |
| | Duncton, Matthew. (2015). Academic Press, 205- |
| | 219. |
| RRRRWW-NH2 | Brito, R., et al (2014). Cells, 3(2), 517-545. |
| | Himmel, H. M., et al. (2002). The Journal of |
| | pharmacology and experimental therapeutics, |
| | 301(3), 981-986. |
| Methoctramine | Brito, R., et al. (2014). Cells, 3(2), 517-545. |
| | Mellor, I. R., et al. (2004). European Journal of |
| | Pharmacology, 505(1-3), 37-50. |
| AG-489 | Brito, R., et al. (2014). Cells, 3(2), 517-545. |
| | Kitaguchi, T., et al. (2005). Biochemistry, 44(47), |
| | 15544-15549. |
| AG-505 | Brito, R., et al. (2014). Cells, 3(2), 517-545. |
| | Kitaguchi, T., et al. (2005). Biochemistry, 44(47), |
| | 15544-15549. |
| DD-161515 | Brito, R., et al. (2014). Cells, 3(2), 517-545. |
| [N-[2-(2-(N- | Garcia-Martinez, C., et al. (2002). Proceedings of |
| methylpyrrolidinyl)ethyl]glycyl]- | the National Academy of Sciences of the United |
| [N-[2,4- | States of America, 99(4), 2374-2379. |
| dichlorophenethyl]glycyl]-N- | |
| (2,4-dichlorophenethyl)glycinamide] | |
| DD-191515 | Brito, R., et al. (2014). Cells, 3(2), 517-545. |
| [[N-[3-(N,N- | Garcia-Martinez, C., et al. (2002). Proceedings of |
| diethylamino)propyl]glycyl]- | the National Academy of Sciences of the United |
| [N-[2,4- | States of America, 99(4), 2374-2379. |
| dichlorophenethyl]glycyl]-N- | |
| (2,4-dichlorophenethyl)glycinamide] | |
| A-784168 | Brito, R., et al. (2014). Cells, 3(2), 517-545. |
| [1-[3-(trifluoromethyl)pyridin-2- | Cui, M., et al. (2006). The Journal of Neuroscience: |
| yl]-N-[4- | the Official Journal of the Society for |
| (trifluoromethylsulfonyl)phenyl]- | Neuroscience, 26(37), 9385-9393. |
| 1,2,3,6- | |
| tetrahydropyridine-4-carboxamide] | |
| A-795614 | Brito, R., et al. (2014). Cells, 3(2), 517-545. |
| [N-1H-indazol-4-yl-N′-[(1R)- | Cui, M., et al. (2006). The Journal of Neuroscience: |
| 5-piperidin-1-yl-2,3-dihydro- | the Official Journal of the Society for |
| 1H-inden-1-yl]urea] | Neuroscience, 26(37), 9385-9393. |
| AMG-0347 | Brito, R., et al. (2014). Cells, 3(2), 517-545. |
| [(E)-N-(7-hydroxy-5,6,7,8- | Steiner, A. A., et al. (2007). The Journal of |
| tetrahydronaphthalen-1-yl)-3- | Neuroscience: the Official Journal of the Society |
| (2-(piperidin-1-yl)-6- | for Neuroscience, 27(28), 7459-7468. |
| (trifluoromethyl)pyridin-3- | |
| yl)acrylamide] | |
| AMG-517 | Brito, R., et al. (2014). Cells, 3(2), 517-545. |
| [N-(4-[6-(4-trifluoromethyl- | Gavva, N. R., et al. (2007). The Journal of |
| phenyl)-pyrimidin-4-yloxy]- | pharmacology and experimental |
| benzothiazol-2-yl)-acetamide I] | therapeutics, 323(1), 128-137. |
| Pentapeptide- | Wandrey, F., et al. (2017). Personal Care Europe, |
| 59/SensAmone P5 | 117-119. |
| RTX [resiniferatoxin] | Wahl, P., et al. (2001). Molecular Pharmacology, |
| | 59(1), 9-15. |
| | Chou, M. Z., et al. (2004). Biochemistry, 43(9), |
| | 2501-2511. |
| SYMSITIVE 1609 [4-tertiary | U.S. Pat. No. 9,155,714 |
| butyl cyclohexane] | Kueper, T. et al. (2010) Exp Dermatol 19(11): |
| | 980-986. |

In some embodiments, the aqueous topical formulations disclosed herein comprise at least one TRPV1 antagonist chosen from Table A.

Certain marine natural products, including from anemones or microalgae, exhibit TRPV1 activity. For example, sea anemones produce a small protein (56 amino acids) called APHC1 that inhibits TRPV1, and a smaller pentapeptide (RRRFV) that retains TRPV1 activity has been designed, based on APHC1, for improved formulation and sustainability. A commercially available product from Mibelle AG Biochemistry, SensAmone P5, contains this small pentapeptide encapsulated in a lipid-based carrier system. The carrier system protects the pentapeptide from degradation and improves skin penetration, and can release the pentapeptide upon pressure during application or being pumped from a packaging container. The INCI description for SensAmone P5 is Pentapeptide-59 (and) Hydrogenated Lecithin (and) Butyrospermum Parkii (Shea) Butter (and) Phenethyl Alcohol (and) Ethylhexylglycerin (and) Maltodextrin (and) Aqua/Water. Other commercially available products that may affect the production of and/or inhibit TRPV1 are present in Mariliance™ and Sensityl™ from Givaudan, both including an extract originating from microalgae. Mariliance includes an extract of a red microalgae, Rhodosorus Marinus, and Sensityl includes an extract of a diatom, Phaeodactylum tricornutum.

Certain plant extracts have been found to exhibit TRPV1 activity. For example, extracts of plant materials of Mangifera indica, Fumaria officinalis, and Rumex japonicus show TRPV1 antagonistic effects. EP 2700431A1. Additionally, extracts of guaiacwood from the Zygophyllaceae plant family, such as extracts of Guaiaicum officinale and Guaiacum sanctum, show TRPV1 antagonistic effects. US2013/0315843. Terpenes, which are produced by plants including conifers, modified terpenes, and terpene derivatives, including borneol, bornyl acetate; and isobornyl isobutyrate, can be isolated from plants in the Heterotheca, Artemisia, Callicarpa and Dipterocarpaceae families, Blumea balsamifera, and Kaempferia galanga, and also show TRPV1 antagonistic effects. Id. Piperitone is a monoterpene ketone that can be isolated from plants in the Cymbopogon, Andropogon, and Mentha families, and shows TRPV1 antagonistic effects. Id. Hydroxy-citronellal can be isolated from species of lemongrass (Cymbopogon), and plants that produce citronella oil, and also show TRPV1 antagonistic effects. Id. Extracts from citrus plants, such as Citrus sinesis and Citrus bergamia, can include jasminone (2-(trans-2-pentenyl)cyclopentanone) and dihydrojasmone (3-methyl-2-pentylcyclopent-2-en-1-one), which show TRPV1 antagonistic effects. Id. Extracts from vanilla beans, which are produced by orchids of the Vanilla family, including Vanilla planifolia, Vanilla tahitensis, and Vanilla pompona, include vanillin. A vanillin derivative, vanillin propylene glycol acetate, shows TRPV1 antagonistic effects. Id.

In at least one embodiment, the aqueous topical formulation includes at least one TRPV1 antagonist that is chosen from the following: JYL-1421 [N-(4-tert-butylbenzyl)-N′-[3-fluoro-4-(methylsulfonylamino)benzyl]thiourea]; KJM429 [N-(4-tert-butylbenzyl)-N′-[4-(methylsulfonylamino)benzyl]thiourea]; A-425619 [1-isoquinolin-5-yl-3-(4-trifluoromethyl-benzyl)-urea]; BCTC [N-(4-tertiarybutylphenyl)-4-(3-chloropyridin-2-yl)tetrahydropyrazine-1 (2H)-carbox-amide]; JNJ-17203212 [4-(3-trifluoromethylpyridin-2-yl) piperazine-1-carboxylic acid (5-trifluoromethylpyridin-2-yl)amide]; SB-705498 [N-(2-bromophenyl)-N′—[((R)-1-(5-trifluoromethyl-2-pyridyl)pyrrolidin-3-yl)]urea]; SB-366791 [4′-Chloro-3-methoxycinnamanilide]; AMG-9810 [(E)-3-(4-t-butylphenyl)-N-(2,3-dihydrobenzo[b][1,4]dioxin-6-yl)acrylamide]; AMG-2674 [3-Amino-5-[[2-[(2-methoxyethyl)amino]-6-[4-(trifluoromethyl)phenyl]-4-pyrimidinyl]oxy]-2 (1H)-quinoxalinone]; Capsazepine; MK-2295 [6-((R)-4-(6-(4-fluorophenyl)-2-((R)-2-methylpyrrolidin-1-yl)pyrimidin-4-yl)-3-methylpiperazin-1-yl)-5-methylnicotinic acid]; Ruthenium red; RRRRWW-NH2; Methoctramine; AG-489; AG-505; DD-161515 [N-[2-(2-(N-methylpyrrolidinyl)ethyl]glycyl]-[N-[2,4-dichlorophenethyl]glycyl]-N-(2,4-dichlorophenethyl)glycinamide]; DD-191515 [N-[3-(N,N-diethylamino) propyl]glycyl]-[N-[2,4-dichlorophenethyl]glycyl]-N-(2,4-dichlorophenethyl)glycinamide]; A-784168 [1-[3-(trifluoromethyl)pyridin-2-yl]-N-[4-(trifluoromethylsulfonyl)phenyl]-1,2,3,6-tetrahydropyridine-4-carboxamide]; A-795614 [N-1H-indazol-4-yl-N′-[(1R)-5-piperidin-1-yl-2,3-dihydro-1H-inden-1-yl]urea]; AMG-0347 [(E)-N-(7-hydroxy-5,6,7,8-tetrahydronaphthalen-1-yl)-3-(2-(piperidin-1-yl)-6-(trifluoromethyl)pyridin-3-yl)acrylamide]; AMG-517 [N-(4-[6-(4-trifluoromethyl-phenyl)-pyrimidin-4-yloxy]-benzothiazol-2-yl)-acetamide I]; Pentapeptide-59; Mariliance; Sensityl; resiniferatoxin, SYMSITIVE 1609 [4-tertiary butyl cyclohexane], Apritone [2-[(2E)-3,7-dimethyl-2,6-octadien-1-yl]cyclopentanone]; (−)-bornyl acetate; hydroxycitronellal [(7-hydroxy-3,7-dimethyloctanal]; methyl N,N-dimethylanthranilate; 2-ethoxy-3-ethylpyrazine; L-piperitone; isobornyl isobutyrate; 4-acetoxy-2,5-dimethyl-3 (2H)-furanone; tripropylamine; dihydrojasmone [3-methyl-2-pentylcyclopent-2-en-1-one]; 1-methyl-2-pyrole carboxaldehyde; 3-octyl acetate; 2-methylbutyl isovalerate; jasminone [2-(trans-2-pentenyl)cyclopentanone]; piperonyl isobutyrate; phenoxyethyl propionate; vanillin propylene glycol acetate; octenyl cyclopentanone; butyl isobutyrate; guaiacwood oil; tetrahydro-4-methyl-2-(2-methyl-1-propenyl)-2H pyran; and 4-tert-butyl cyclohexanol.

In at least one embodiment, the aqueous topical formulation includes a TRPV1 antagonist that is Pentapeptide 59/SensAmone P5. In at least one embodiment, the aqueous topical formulation includes a TRPV1 antagonist that is a structural derivative or analog of APHC1. In at least one embodiment, the aqueous topical formulation includes a TRPV1 modulator that is Pentapeptide 59/SensAmone P5. In at least one embodiment, the TRPV1 antagonist comprises Pentapeptide 59/SensAmone P5. In at least one embodiment, the TRPV1 modulator comprises Pentapeptide 59/SensAmone P5. In at least one embodiment, the aqueous topical formulation includes at least one of Pentapeptide 59/SensAmone P5, Mariliance™ and Sensityl™. In at least one embodiment, the aqueous topical formulation includes a marine natural product derived from a sea anemone or microalgae. In at least one embodiment, the aqueous topical formulation includes a TRVP1 antagonist that comprises a microalgae extract. In at least one embodiment, the aqueous topical formulation includes a TRVP1 antagonist that comprises a red microalgae extract. In at least one embodiment, the aqueous topical formulation includes a TRVP1 antagonist that comprises a diatom extract. In at least one embodiment, the aqueous topical formulation includes a TRVP1 antagonist that comprises an extract of Rhodosorus Marinus. In at least one embodiment, the aqueous topical formulation includes a TRVP1 antagonist that comprises an extract of Phaeodactylum tricornutum. In at least one embodiment, the aqueous topical formulation includes a TRVP1 antagonist and a microalgae extract. In at least one embodiment, the aqueous topical formulation includes a TRVP1 antagonist that comprises a marine natural product. In at least one embodiment, the aqueous topical formulation includes a TRVP1 antagonist that is derived from a sea anemone.

In at least one embodiment, the aqueous topical formulation includes at least one TRPV1 antagonist comprising Mangifera indica extract. In at least one embodiment, the aqueous topical formulation includes at least one TRPV1 antagonist comprising Fumaria officinalis extract. In at least one embodiment, the aqueous topical formulation includes at least one TRPV1 antagonist comprising Rumex japonicus extract. In at least one embodiment, the aqueous topical formulation includes at least one TRPV1 antagonist comprising at least one of guaiacwood extract, Guaiaicum officinale extract, and guaiacum sanctum extract. In at least one embodiment, the aqueous topical formulation includes at least one TRPV1 antagonist comprising guaiacwood extract. In at least one embodiment, the aqueous topical formulation includes at least one TRPV1 antagonist comprising Blumea balsamifera extract. In at least one embodiment, the aqueous topical formulation includes at least one TRPV1 antagonist comprising Kaempferia galanga extract. In at least one embodiment, the aqueous topical formulation includes at least one TRPV1 antagonist comprising lemongrass extract. In at least one embodiment, the aqueous topical formulation includes at least one TRPV1 antagonist comprising citrus extract. In at least one embodiment, the aqueous topical formulation includes at least one TRPV1 antagonist comprising citronella oil extract. In at least one embodiment, the aqueous topical formulation includes at least one TRPV1 antagonist comprising citrus oil extract. In at least one embodiment, the aqueous topical formulation includes at least one TRPV1 antagonist comprising Citrus sinesis extract. In at least one embodiment, the aqueous topical formulation includes at least one TRPV1 antagonist comprising Citrus bergamia extract. In at least one embodiment, the aqueous topical formulation includes at least one TRPV1 antagonist comprising Vanilla planifolia extract. In at least one embodiment, the aqueous topical formulation includes at least one TRPV1 antagonist comprising Vanilla tahitensis extract. In at least one embodiment, the aqueous topical formulation includes at least one TRPV1 antagonist comprising Vanilla pompona extract. In at least one embodiment, the aqueous topical formulation includes at least one TRPV1 antagonist comprising vanilla extract.

In at least one embodiment, the aqueous topical formulation includes at least one TRPV1 antagonist and Mangifera indica extract. In at least one embodiment, the aqueous topical formulation includes at least one TRPV1 antagonist and Fumaria officinalis extract. In at least one embodiment, the aqueous topical formulation includes at least one TRPV1 antagonist and Rumex japonicus extract. In at least one embodiment, the aqueous topical formulation includes at least one TRPV1 antagonist and at least one of guaiacwood extract, Guaiaicum officinale extract, and guaiacum sanctum extract. In at least one embodiment, the aqueous topical formulation includes at least one TRPV1 antagonist and guaiacwood extract. In at least one embodiment, the aqueous topical formulation includes at least one TRPV1 antagonist and Blumea balsamifera extract. In at least one embodiment, the aqueous topical formulation includes at least one TRPV1 antagonist and Kaempferia galanga extract. In at least one embodiment, the aqueous topical formulation includes at least one TRPV1 antagonist and lemongrass extract. In at least one embodiment, the aqueous topical formulation includes at least one TRPV1 antagonist and citrus extract. In at least one embodiment, the aqueous topical formulation includes at least one TRPV1 antagonist and citronella oil extract. In at least one embodiment, the aqueous topical formulation includes at least one TRPV1 antagonist and citrus oil extract. In at least one embodiment, the aqueous topical formulation includes at least one TRPV1 antagonist and citrus sinesis extract. In at least one embodiment, the aqueous topical formulation includes at least one TRPV1 antagonist and Citrus bergamia extract. In at least one embodiment, the aqueous topical formulation includes at least one TRPV1 antagonist and Vanilla planifolia extract. In at least one embodiment, the aqueous topical formulation includes at least one TRPV1 antagonist and Vanilla tahitensis extract. In at least one embodiment, the aqueous topical formulation includes at least one TRPV1 antagonist and Vanilla pompona extract. In at least one embodiment, the aqueous topical formulation includes at least one TRPV1 antagonist and vanilla extract.

In at least one embodiment, the aqueous topical formulation includes at least one TRPV1 antagonist that is a plant extract chosen from the following: Mangifera indica extract, Fumaria officinalis extract, Rumex japonicus extract, guaiacwood extract, Guaiaicum officinale extract, guaiacum sanctum extract, Blumea balsamifera extract, Kaempferia galanga extract, lemongrass extract, citronella oil extract, citrus oil extract, Citrus sinesis extract, Citrus bergamia extract, Vanilla planifolia extract, Vanilla tahitensis extract, and Vanilla pompona extract. In at least one embodiment, the aqueous topical formulation includes at least one TRPV1 antagonist and at least one plant extract chosen from the following: Mangifera indica extract, Fumaria officinalis extract, Rumex japonicus extract, guaiacwood extract, Guaiaicum officinale extract, guaiacum sanctum extract, Blumea balsamifera extract, Kaempferia galanga extract, lemongrass extract, citronella oil extract, citrus oil extract, Citrus sinesis extract, Citrus bergamia extract, Vanilla planifolia extract, vanilla tahitensis extract, and Vanilla pompona extract.

In some embodiments, a transient receptor potential ankyrin (TRPA) antagonist may be included in the aqueous topical formulations described herein. Mammals have only one member of the TRPA ion channel, TRPA1, which is expressed in sensory neurons, epithelial cells, and hair cells. Talavera et al, Physiol Rev 100, 725-803 (2020).

Nonlimiting examples of TRPA antagonists include isobornyl isobutryate, which is a terpene derivative; phloretin, which can be isolated from Prunus mandschurica and apple tree leaves; 3,3,5-trimethylcyclohexanol; cinnamon bark oil, which can be isolated from species of trees in the Cinnamomum family; γ-dodecalactone, which can be isolated from strawberries; vanillic acid, which can be isolated from Angelica sinesis and Acai oil, and is an oxidized form of vanillin; γ-methyl decalactone; trans, trans-2,4-nonadienal; 4-allyl-2,6-dimethoxyphenol; o-methoxycinnamaldehyde; 4-methyl-2-phenyl-2 pentenal (mix of cis and trans); 2-methoxy-4-propyl-phenol; methyl 2-methoxy-benzoate; δ-tetradecalactone; 1-methyl-2-pyrole carboxaldehyde; 3,3,5-trimethylcyclohexanol; N-(2-hydroxyethyl) lactamide; 2-(3-phenylpropyl) tetrahydrofuran; anisyl butyrate; methyl-4-phenyl butyrate; 3-heptyldihydro-5-methyl-2 (3H)-furanone; 3-acetylsulfanylhexyl acetate; 3-methyl-5-propyl-2-cyclohexen-1-one; bornyl valerate; citronellyl acetate, which can be obtained from citronella oil; (2S,5S,6S)-6-)hydroxy-dihydrotheaspirane; and trans-2-hexenal. In at least one embodiment, the aqueous topical formulation includes a TRVP1 antagonist and a TRPA antagonist. In at least one embodiment, the aqueous topical formulation includes at least one TRPA antagonist that is chosen from a terpene derivative, Prunus mandschurica extract, apple tree leaf extract, cinnamon bark extract, strawberry extract, Angelica sinesis extract, citronella oil extract, and acai oil extract. In at least one embodiment, the aqueous topical formulation includes a TRVP1 antagonist that is also a TRPA antagonist. In at least one embodiment, the aqueous topical formulation includes a TRPA antagonist instead of a TRPV1 antagonist. In at least one embodiment, the aqueous topical formulation includes a TRPA antagonist in addition to a TRPV1 antagonist

In at least one embodiment, the aqueous topical formulation includes at least one TRPV1 antagonist that is encapsulated in a lipid-based carrier system. For example, the lipid-based carrier system may include a glycol such as pentylene glycol, propane-1,3-diol, or propylene glycol (also known as propane-1,2-diol); glycerin or a glyceryl derivative such as glyceryl laurate or ethylhexylglycerin; or a polyethylene glycol having a molecular weight of less than about 50,000. The lipid-based carrier system may include lipids or other oil-based components that can act as a matrix in addition to an encapsulating component or shell. The matrix can include coconut oil, sunflower oil, safflower oil, or a lecithin. In at least one embodiment, the aqueous topical formulation includes a TRPV1 antagonist that is encapsulated in a lipid-based carrier system. In at least one embodiment, the lipid-based carrier system comprises shea butter. In at least one embodiment, the aqueous topical formulation includes at least one TRPV1 antagonist and shea butter. In at least one embodiment, the aqueous topical formulation includes at least one TRPV1 antagonist and shea butter extracted or derived from Butyrospermum parkii. In at least one embodiment, the aqueous topical formulation includes at least one TRPV1 antagonist and shea butter extracted or derived from Vitellaria paradoxa. In at least one embodiment, the aqueous topical formulation includes at least one TRPV1 antagonist, shea butter, hydrogenated lecithin, phenethyl alcohol, ethylhexylglycerin, and maltrodextrin.

The aqueous topical formulations disclosed herein can further comprise at least one cooling agent which may elicit a cooling sensation upon contact with skin, such as menthol, menthoxypropanediol, and isopulegol. Menthol is a TRPM8 agonist and can provide a cooling response due to activation of the TRPM8 receptor. Other TRPM8 agonists include borneol, linalool, geraniol, hydroxy-citronellal, icilin, WS-12, and p-menthane-3,8-diol (PMD). Borneol, linalool, geraniol, hydroxy-citronellal and PMD are terpenes, modified terpenes, or terpene derivatives. Terpenes are produced by plants, including by conifers. For example, borneol can be isolated from plants in the Heterotheca, Artemisia, Callicarpa and Dipterocarpaceae families, Blumea balsamifera, and Kaempferia galanga; linalool can be isolated from coriander including Coriandrum sativum, citrus including Citrus sinensis, and from species in the lavender family (Lavandula) including Lavandula angustifolia, bay laurel family, basil family (Ocimum basilicum) including sweet basil and holy basil (Ocimum tenuiflorum), and mint family including Mentha piperita (peppermint), Mentha spicata (spearmint), and Mentha haplocalyx (bohe); geraniol can be isolated from citronella oil, rose oil, and palmarosa oil, as well as from species in the geranium genus; hydroxy-citronellal can be isolated from species of lemongrass (Cymbopogon), and plants that produce citronella oil; and PMD can be isolated from Corymbia citriodora.

In at least one embodiment, the aqueous topical formulation includes a TRPV1 antagonist and menthoxypropanediol. In at least one embodiment, the aqueous topical formulation comprises a TRPV1 antagonist and menthoxypropanediol. In at least one embodiment, the aqueous topical formulation comprises a TRPV1 antagonist and menthol. In at least one embodiment, the aqueous topical formulation comprises a TRPV1 antagonist and isopulegol. In at least one embodiment, the aqueous topical formulation comprises at least one TRPV1 antagonist and at least one of menthol, menthoxypropanediol, and isopulegol. In at least one embodiment, the aqueous topical formulation comprises at least one TRPV1 antagonist and at least one TRPM8 agonist. In at least one embodiment, the aqueous topical formulation comprises at least one TRPV1 antagonist and at least one of a terpene, modified terpene, or a terpene derivative. In at least one embodiment, the aqueous topical formulation comprises at least one TRPV1 antagonist and at least one TRPM8 agonist chosen from borneol, linalool, geraniol, hydroxy-citronellal, icilin, WS-12, and PMD. In at least one embodiment, the aqueous topical formulation comprises at least one TRPV1 antagonist and at least one plant extract chosen from conifer extract, an extract from a plant in the Heterotheca family, an extract from a plant in the Artemisia family, an extract from a plant in the Callicarpa family, an extract from a plant in the Dipterocarpaceae family, Blumea balsamifera extract, Kaempferia galanga extract, coriander extract, Coriandrum sativum extract, an extract from a plant in the citrus family, Citrus sinensis extract, lavender extract, bay laurel extract, basil extract, mint extract, citronella oil extract, rose oil extract, palmarosa oil extract, geranium extract, an extract from a plant in the Cymbopogon family, and Corymbia citriodora extract. In at least one embodiment, the aqueous topical formulation comprises at least one TRPV1 antagonist and at least one plant extract chosen from conifer extract, Blumea balsamifera extract, Kaempferia galanga extract, coriander extract, Coriandrum sativum extract, Citrus sinensis extract, lavender extract, bay laurel extract, basil extract, mint extract, citronella oil extract, rose oil extract, palmarosa oil extract, geranium extract, Cymbopogon citratus extract, and Corymbia citriodora extract.

In at least one embodiment, the aqueous topical formulation comprises a TRPV1 antagonist and conifer extract. In at least one embodiment, the aqueous topical formulation comprises a TRPV1 antagonist and Blumea balsamifera extract. In at least one embodiment, the aqueous topical formulation comprises a TRPV1 antagonist and Kaempferia galanga extract. In at least one embodiment, the aqueous topical formulation comprises a TRPV1 antagonist and coriander extract. In at least one embodiment, the aqueous topical formulation comprises a TRPV1 antagonist and Coriandrum sativum extract. In at least one embodiment, the aqueous topical formulation comprises a TRPV1 antagonist and Citrus sinesis extract. In at least one embodiment, the aqueous topical formulation comprises a TRPV1 antagonist and lavender extract. In at least one embodiment, the aqueous topical formulation comprises a TRPV1 antagonist and Lavandula angustifolia extract. In at least one embodiment, the aqueous topical formulation comprises a TRPV1 antagonist and bay laurel extract. In at least one embodiment, the aqueous topical formulation comprises a TRPV1 antagonist and basil extract. In at least one embodiment, the aqueous topical formulation comprises a TRPV1 antagonist and Ocimum basilicum extract. In at least one embodiment, the aqueous topical formulation comprises a TRPV1 antagonist and sweet basil extract. In at least one embodiment, the aqueous topical formulation comprises a TRPV1 antagonist and Ocimum tenuiflorum extract.

In at least one embodiment, the aqueous topical formulation comprises a TRPV1 antagonist and Mentha piperita extract. In at least one embodiment, the aqueous topical formulation comprises a TRPV1 antagonist and Mentha spicata extract. In at least one embodiment, the aqueous topical formulation comprises a TRPV1 antagonist and Mentha haplocalyx extract. In at least one embodiment, the aqueous topical formulation comprises a TRPV1 antagonist and mint extract. In at least one embodiment, the aqueous topical formulation comprises a TRPV1 antagonist and citronella oil extract. In at least one embodiment, the aqueous topical formulation comprises a TRPV1 antagonist and rose oil extract. In at least one embodiment, the aqueous topical formulation comprises a TRPV1 antagonist and palmerosa oil extract. In at least one embodiment, the aqueous topical formulation comprises a TRPV1 antagonist and geranium extract. In at least one embodiment, the aqueous topical formulation comprises a TRPV1 antagonist and Cymbopogon citratus extract. In at least one embodiment, the aqueous topical formulation comprises a TRPV1 antagonist and Corymbia citriodora extract.

The aqueous topical formulations disclosed herein can further comprise at least one humectant and/or moisturizing agent, such as betaine; aloe; a glycol or glycol derivative such as polyethylene glycol, pentylene glycol, propane-1,3-diol, or propylene glycol (also known as propane-1,2-diol); glycerin or a glyceryl derivative such as glyceryl laurate or ethylhexylglycerin; sorbitol or a sorbitol derivative such as a sorbitan oleate decylglucoside polymer; at least one fatty acid such as coconut oil; lecithin; or honey. The lecithin may be hydrogenated.

In at least one embodiment, the aqueous topical formulation includes at least one TRPV1 antagonist and at least one of betaine, aloe, pentylene glycol, propylene glycol, glyceryl laurate, ethylhexylglycerin, glycerin, a sorbitan oleate decylglucoside polymer, lecithin, and coconut oil. In at least one embodiment, the aqueous topical formulation comprises at least one TRPV1 antagonist, betaine, pentylene glycol, ethylhexylglycerin, glycerin, a sorbitan oleate decylglucoside polymer, lecithin, and coconut oil. In at least one embodiment, the aqueous topical formulation comprises at least one TRPV1 antagonist, ethylhexylglycerin, glycerin, hydrogenated lecithin, propylene glycol, and coconut oil. In at least one embodiment, the aqueous topical formulation comprises a TRPV1 antagonist, ethylhexylglycerin, pentylene glycol, hydrogenated lecithin, and glycerin. In at least one embodiment, the aqueous topical formulation includes at least one TRPV1 antagonist, pentylene glycol, ethylhexylglycerin, glycerin, a sorbitan oleate decylglucoside polymer, hydrogenated lecithin, propylene glycol, and aloe.

The aqueous topical formulations disclosed herein can further include skin-repairing components such as sodium hyaluronate or other salt or derivative of hyaluronic acid. In at least one embodiment, the aqueous topical formulation comprises at least one TRPV1 antagonist and sodium hyaluronate. In at least one embodiment, the aqueous topical formulation comprises at least one TRPV1 antagonist and a hyaluronate salt. In at least one embodiment, the aqueous topical formulation comprises sodium hyaluronate. In at least one embodiment, the aqueous topical formulation comprises at least one TRPV1 antagonist and sodium hyaluronate.

The aqueous topical formulations disclosed herein can further include other components that are beneficial to skin. For example, the formulation can include at least one collagen promoting agent, such as a palmitoyl-lysyl-valyl-lysine acetate salt, which is available in the commercially available product Syn-Coll®. In at least one embodiment, the aqueous topical formulation comprises at least one TRPV1 antagonist and palmitoyl-lysyl-valyl-lysine bistrifluoracetate salt.

The aqueous topical formulations disclosed herein can further include other components that are beneficial to skin. For example, the formulation can include at least one amino acid, such as arginine or a salt thereof. In at least one embodiment, the aqueous topical formulation comprises at least one TRPV1 antagonist and at least of arginine and an arginine salt. In at least one embodiment, the aqueous topical formulation comprises at least one TRPV1 antagonist and arginine.

The aqueous topical formulations disclosed herein can further comprise at least one vitamin or a salt thereof, such as vitamin E, vitamin B, or any form of vitamins E or B. For example, the B vitamins include thiamine (vitamin B1), riboflavin (vitamin B2), niacin (vitamin B3), pantothenic acid (vitamin B5), vitamin B6, biotin (vitamin B7), folic acid (vitamin B9), and cobalamin (vitamin B12); and vitamin E includes four tocopherols, including tocopherol and tocopheryl acetate, and four tocotrienols.

In at least one embodiment, the aqueous topical formulation comprises a TRPV1 antagonist and at least one form of vitamin E or vitamin B. In at least one embodiment, the aqueous topical formulation comprises a TRPV1 antagonist and at least one form of vitamin E. In at least one embodiment, the aqueous topical formulation comprises a TRPV1 antagonist and at least one form of vitamin B. In at least one embodiment, the aqueous topical formulation comprises a TRPV1 antagonist and at least one form of each of vitamin E and vitamin B. In at least one embodiment, the aqueous topical formulation comprises a TRPV1 antagonist and at least one of tocopherol and tocopheryl acetate. In at least one embodiment, the aqueous topical formulation comprises a TRPV1 antagonist and tocopheryl acetate. In at least one embodiment, the aqueous topical formulation comprises a TRPV1 antagonist and tocopherol.

The aqueous topical formulations disclosed herein can further include at least one component that is beneficial to skin and that is based on, or include, seed or plant extracts. For example, extracts that have anti-inflammatory or anti-irritant activity, such as aloe vera juice, Chamomilla recutita flower extract, witch hazel leaf extract, or an oat kernel extract including oat kernel flour, may be included. Witch hazel is a genus of flowering plants in the Hamamelidaceae family, and includes Hamamelis virginiana. Plants that contain phenolic alkaloids called avenanthramides includes oats, such as Avena sativa, and avenanthramides have anti-inflammatory and antioxidant activity. Oxalic-acid containing seeds or plants, or extracts thereof, may be used as an anti-oxidant, such as quinoa or other plants of the Oxalis, Chenopodium or Amaranthaceae families. The seed or plant extract may be solubilized in an oil or other solvent, such as glycerin. Fruit extracts with mild cleansing properties, such as Sapindus trifoliatus fruit extract, may be included. Punica granatum (pomegranate) and Myrciaria dubia (camu-camu fruit) contain high amounts of vitamin C, and their extracts may be included. Houttuynia cordata (fish mint) extracts, including of the leaves or flowers, may be included. Edible seaweed extracts, including of the Ulva (sea lettuce) family such as Ulva compressa (formerly known as Enteromorpha compressa), may be included.

In at least one embodiment, the aqueous topical formulation comprises a TRPV1 antagonist and an extract of at least one of aloe, chamomile, witch hazel, oats, quinoa, Sapindus trifoliatus fruit, pomegranate, Myrciaria dubia, Houttuynia cordata, or Ulva compressa. In at least one embodiment, the aqueous topical formulation comprises a TRPV1 antagonist and at least one of an aloe extract, chamomile extract, witch hazel extract, oat kernel extract, oat kernel flour, hydrolyzed quinoa, or Sapindus trifoliatus fruit extract. In at least one embodiment, the aqueous topical formulation comprises a TRPV1 antagonist and aloe or aloe extract. In at least one embodiment, the aqueous topical formulation comprises a TRPV1 antagonist and Aloe barbadensis leaf juice. In at least one embodiment, the aqueous topical formulation comprises a TRPV1 antagonist and a chamomile flower extract. In at least one embodiment, the aqueous topical formulation comprises a TRPV1 antagonist and a witch hazel extract. In at least one embodiment, the aqueous topical formulation comprises a TRPV1 antagonist and an oat kernel extract. In at least one embodiment, the aqueous topical formulation comprises a TRPV1 antagonist and Avena sativa flour. In at least one embodiment, the aqueous topical formulation comprises a TRPV1 antagonist and hydrolyzed quinoa. In at least one embodiment, the aqueous topical formulation comprises a TRPV1 antagonist and a Sapindus trifoliatus fruit extract. In at least one embodiment, the aqueous topical formulation comprises a TRPV1 antagonist and witch hazel leaf extract. In at least one embodiment, the aqueous topical formulation comprises a TRPV1 antagonist and pomegranate extract. In at least one embodiment, the aqueous topical formulation comprises a TRPV1 antagonist and Myrciaria dubia extract. In at least one embodiment, the aqueous topical formulation comprises a TRPV1 antagonist and Houttuynia cordata extract. In at least one embodiment, the aqueous topical formulation comprises a TRPV1 antagonist and Ulva compressa extract. In at least one embodiment, the aqueous topical formulation comprises a TRPV1 antagonist, Chamomilla recutita extract, Avena sativa extract, and Sapindus trifoliatus fruit extract. In at least one embodiment, the aqueous topical formulation comprises a TRPV1 antagonist, aloe barbadensis leaf juice, and hydrolyzed quinoa.

The aqueous topical formulations disclosed herein can further include components that have a pleasant or beneficial scent. These components may be based on, or include, seed or plant extracts. For example, extracts that are chocolate scented, such as Akebia quinate or extracts from the cocoa bean plant (Theobroma cacao); melon scented, such as cucumber extract, including Cucumis sativus fruit extract; coconut scented, such as coconut extracts such as from Cocos nucifera, including coconut, coconut oil, coconut endosperm, coconut fruit juice, or coconut water; or extracts and/or components that are floral scented, such as Rosa damascena flowers, Jasminum officianale (jasmine) flowers, Cananga odorata (ylang-ylang) flowers, or phenethyl alcohol, may be included. Similarly, plant extracts that have an herbal scent, including from plants of the Agathosma plant family such as Agathosma betulina (buchu leaf), from plants of the Mentha (mint) family such as Mentha canadensis, from plants of the Asteraceae family such as Eclipta prostrate (false daisy), Calendula (marigold), and Silybum marianum (milk thistle), from Cydonia oblonga (quince tree), from Oenothera biennis (evening primrose), from Lepidium meyenii (maca), from Ulmus rubra (slippery elm), from Olea europaea (olive), from Acmella oleracea, from Humulus lupulus (hops), and from Nicotiana sylvestris (flowering tobacco) may be included.

Extracts from plants, including of the flower, seed, fruit, petals, and/or stem, that are commonly associated with various traditional medicines may be included in the aqueous topical formulations discloses herein, including from the Vaccinium oxycoccus (cranberry) family such as Vaccinium macrocarpon, from the Opuntia (prickly pear cactus) family, from Gymnema sylvestre (gurmar plant), from the Portulaca (purslane) family, from the Rosa (rose) family such as Rosa penduline (Alpine rose), from Ceratonia siliqua (carob), from the Prunus family such as Prunus amygdalus (almond tree), Prunus dulcis (sweet almond), and Prunus armeniaca (apricot), from Ginkgo biloba, from avocado plants such as Persea americana, from Camellia Sinensis (tea plant), from the Melaleuca (myrtle) family such as Eucalyptus, from Linum usitatissimum (linseed), from Cyamopsis tetragonoloba (guar), from the Hippophae (sea buckthorn) family, from Helianthus annuus (sunflower), from Simmondsia chinensis (jojoba), from Limnanthes alba (white meadowform), from the Sesamum (sesame) family such as Sesamum Indicum, from Azadirachta indica (Indian lilac), from Moringa oleifera (horseradish tree), from Cedrus atlantica (Atlas cedar), from the Calophyllum family such as Calophyllum inophyllum and Calophyllum tacamahaca, from the Daucus carota (carrot) family such as Daucus Carota Sativa, from Euphorbia cerifera (also known as Euphorbia antisyphilitica) (candelilla), from Copernicia prunifera (carnauba palm), from Echinacea purpurea (coneflower), from Zea mays (corn), from Quercus infectoria (manjakani), from the Pueraria family such as arrowroot, from Eurocoma longifolia (longjack), from Trigonella foenum-graecum (fenugreek), from the Salvia family such as Salvia rosmarinus (rosemary), Salva officinalis (common sage), and Salvia sclarea (clary sage), from Oryza sativa (Asian rice), from Zingiber officinale (ginger), from Glycyrriza glabra (licorice), Matricaria recutita (German chamomile), from the Elaeis (oil palm) family, from Manihot esculenta (tapioca), from Vitis vinifera (grape), from the Corylus family such as Corylus avellana (hazelnut), from Turnera diffusa (damiana), from Viola sororia (common violet), from the Podocarpus family such as Podocarpus totara, from the Rubus (raspberry) family such as Rubus idaeus and Rubus strigosus, from the Baptisia (wild indigo) family such as Baptisia australis, from Carthamus tinctorius (safflower), and from the Malus (apple) family.

In at least one embodiment, the aqueous topical formulation comprises a TRPV1 antagonist and at least one of phenyethyl alcohol, Akebia quinate extract, Theobroma cacao extract, cucumber extract, coconut extract, Rosa damascena extract, jasmine extract, Cananga odorata extract, Agathosma betulina extract, Mentha canadensis extract, Eclipta prostrate extract, calendula extract, Silybum marianum extract, quince extract, Oenothera biennis extract, Lepidium meyenii extract, Ulmus rubra extract, olive tree extract, Acmella oleracea extract, Humulus lupulus extract, Nicotiana sylvestris extract, Vaccinium oxycoccus extract, Vaccinium macrocarpon extract, prickly pear cactus extract, Gymnema sylvestre extract, purslane extract, Rosa penduline extract, Ceratonia siliqua extract, Prunus amygdalus extract, Prunus dulcis extract, Prunus armeniaca extract, Ginkgo biloba extract, from avocado extract, Persea americana extract, Camellia sinensis extract, eucalyptus extract, Linum usitatissimum extract, Cyamopsis tetragonoloba extract, sea buckthorn extract, Helianthus annuus extract, Simmondsia chinensis extract, Limnanthes alba extract, Sesamum indicum extract, Azadirachta indica extract, Moringa oleifera extract, Cedrus atlantica extract, calophyllum extract, Calophyllum inophyllum extract, Calophyllum tacamahaca extract, Daucus carota extract, Daucus carota sativa extract, euphorbia cerifera extract, candelilla extract, carnauba palm extract, Echinacea purpurea extract, Zea mays extract, Quercus infectoria extract, arrowroot extract, Eurocoma longifolia extract, Trigonella foenum-graecum extract, salvia extract, rosemary extract, sage extract, clary sage extract, Oryza sativa extract, Zingiber officinale extract, licorice extract, Matricaria recutita extract, oil palm extract, tapioca extract, Vitis vinifera extract, hazelnut tree extract, Turnera diffusa extract, violet extract, Podocarpus totara extract, raspberry extract, Rubus idaeus extract, Rubus strigosus extract, wild indigo extract, safflower extract, or apple extract.

In at least one embodiment, the aqueous topical formulation comprises a TRPV1 antagonist and Akebia quinata. In at least one embodiment, the aqueous topical formulation comprises a TRPV1 antagonist and phenethyl alcohol. In at least one embodiment, the aqueous topical formulation comprises a TRPV1 antagonist and Theobroma cacao extract. In at least one embodiment, the aqueous topical formulation comprises a TRPV1 antagonist and cucumber extract. In at least one embodiment, the aqueous topical formulation comprises a TRPV1 antagonist and Cucumis sativus fruit extract. In at least one embodiment, the aqueous topical formulation comprises a TRPV1 antagonist and coconut. In at least one embodiment, the aqueous topical formulation comprises a TRPV1 antagonist and coconut extract. In at least one embodiment, the aqueous topical formulation comprises a TRPV1 antagonist and coconut liquid endosperm. In at least one embodiment, the aqueous topical formulation comprises a TRPV1 antagonist and coconut water. In at least one embodiment, the aqueous topical formulation comprises a TRPV1 antagonist and coconut fruit juice. In at least one embodiment, the aqueous topical formulation comprises a TRPV1 antagonist and Rosa damascena extract.

In at least one embodiment, the aqueous topical formulation comprises a TRPV1 antagonist and jasmine extract. In at least one embodiment, the aqueous topical formulation comprises a TRPV1 antagonist and Cananga oderata extract. In at least one embodiment, the aqueous topical formulation comprises a TRPV1 antagonist and Agathosma betulina extract. In at least one embodiment, the aqueous topical formulation comprises a TRPV1 antagonist and Mentha canadensis extract. In at least one embodiment, the aqueous topical formulation comprises a TRPV1 antagonist and Eclipta prostate extract. In at least one embodiment, the aqueous topical formulation comprises a TRPV1 antagonist and calendula extract. In at least one embodiment, the aqueous topical formulation comprises a TRPV1 antagonist and Silybum marianum extract. In at least one embodiment, the aqueous topical formulation comprises a TRPV1 antagonist and quince extract. In at least one embodiment, the aqueous topical formulation comprises a TRPV1 antagonist and Oenothera biennis extract. In at least one embodiment, the aqueous topical formulation comprises a TRPV1 antagonist and Lepidum mevenii extract. In at least one embodiment, the aqueous topical formulation comprises a TRPV1 antagonist and Ulmus rubra extract. In at least one embodiment, the aqueous topical formulation comprises a TRPV1 antagonist and olive tree extract. In at least one embodiment, the aqueous topical formulation comprises a TRPV1 antagonist and Acmella oleracea extract. In at least one embodiment, the aqueous topical formulation comprises a TRPV1 antagonist and Humulus lupulus extract. In at least one embodiment, the aqueous topical formulation comprises a TRPV1 antagonist and Nicotiana sylvestris extract.

In at least one embodiment, the aqueous topical formulation comprises a TRPV1 antagonist and cranberry extract. In at least one embodiment, the aqueous topical formulation comprises a TRPV1 antagonist and Vaccinium macrocarpon extract. In at least one embodiment, the aqueous topical formulation comprises a TRPV1 antagonist and prickly pear cactus extract. In at least one embodiment, the aqueous topical formulation comprises a TRPV1 antagonist and Gymnema sylvestre extract. In at least one embodiment, the aqueous topical formulation comprises a TRPV1 antagonist and purslane extract. In at least one embodiment, the aqueous topical formulation comprises a TRPV1 antagonist and rose extract. In at least one embodiment, the aqueous topical formulation comprises a TRPV1 antagonist and Rosa penduline extract. In at least one embodiment, the aqueous topical formulation comprises a TRPV1 antagonist and Ceratonia siliqua extract. In at least one embodiment, the aqueous topical formulation comprises a TRPV1 antagonist and Prunus amygdalus extract. In at least one embodiment, the aqueous topical formulation comprises a TRPV1 antagonist and Prunus dulcis extract. In at least one embodiment, the aqueous topical formulation comprises a TRPV1 antagonist and Prunus armeniaca extract. In at least one embodiment, the aqueous topical formulation comprises a TRPV1 antagonist and Ginkgo biloba extract. In at least one embodiment, the aqueous topical formulation comprises a TRPV1 antagonist and avocado extract. In at least one embodiment, the aqueous topical formulation comprises a TRPV1 antagonist and Persea americana extract.

In at least one embodiment, the aqueous topical formulation comprises a TRPV1 antagonist and Camellia sinensis extract. In at least one embodiment, the aqueous topical formulation comprises a TRPV1 antagonist and myrtle extract. In at least one embodiment, the aqueous topical formulation comprises a TRPV1 antagonist and eucalyptus extract. In at least one embodiment, the aqueous topical formulation comprises a TRPV1 antagonist and Linum usitatissimum extract. In at least one embodiment, the aqueous topical formulation comprises a TRPV1 antagonist and Cyamopsis tetragonoloba extract. In at least one embodiment, the aqueous topical formulation comprises a TRPV1 antagonist and hippophae extract. In at least one embodiment, the aqueous topical formulation comprises a TRPV1 antagonist and sea buckthorn extract. In at least one embodiment, the aqueous topical formulation comprises a TRPV1 antagonist and Helianthus annuus extract. In at least one embodiment, the aqueous topical formulation comprises a TRPV1 antagonist and Simmondsia chinensis extract. In at least one embodiment, the aqueous topical formulation comprises a TRPV1 antagonist and Limnanthes alba extract. In at least one embodiment, the aqueous topical formulation comprises a TRPV1 antagonist and sesame extract. In at least one embodiment, the aqueous topical formulation comprises a TRPV1 antagonist and Sesamum indicum extract.

In at least one embodiment, the aqueous topical formulation comprises a TRPV1 antagonist and Azadirachta indica extract. In at least one embodiment, the aqueous topical formulation comprises a TRPV1 antagonist and Moringa oleifera extract. In at least one embodiment, the aqueous topical formulation comprises a TRPV1 antagonist and Cedrus atlantica extract. In at least one embodiment, the aqueous topical formulation comprises a TRPV1 antagonist and calophyllum extract. In at least one embodiment, the aqueous topical formulation comprises a TRPV1 antagonist and Calophyllum inophyllum extract. In at least one embodiment, the aqueous topical formulation comprises a TRPV1 antagonist and calophyllum tacamahaca extract. In at least one embodiment, the aqueous topical formulation comprises a TRPV1 antagonist and carrot extract. In at least one embodiment, the aqueous topical formulation comprises a TRPV1 antagonist and Daucus carota sativa extract. In at least one embodiment, the aqueous topical formulation comprises a TRPV1 antagonist and Euphorbia cerifera extract. In at least one embodiment, the aqueous topical formulation comprises a TRPV1 antagonist and candelilla extract. In at least one embodiment, the aqueous topical formulation comprises a TRPV1 antagonist and carnauba palm extract. In at least one embodiment, the aqueous topical formulation comprises a TRPV1 antagonist and Echinacea purpurea extract. In at least one embodiment, the aqueous topical formulation comprises a TRPV1 antagonist and corn extract. In at least one embodiment, the aqueous topical formulation comprises a TRPV1 antagonist and Quercus infectoria extract.

In at least one embodiment, the aqueous topical formulation comprises a TRPV1 antagonist and pueraria extract. In at least one embodiment, the aqueous topical formulation comprises a TRPV1 antagonist and arrowroot extract. In at least one embodiment, the aqueous topical formulation comprises a TRPV1 antagonist and Eurocoma longifolia extract. In at least one embodiment, the aqueous topical formulation comprises a TRPV1 antagonist and fenugreek extract. In at least one embodiment, the aqueous topical formulation comprises a TRPV1 antagonist and salvia extract. In at least one embodiment, the aqueous topical formulation comprises a TRPV1 antagonist and rosemary extract. In at least one embodiment, the aqueous topical formulation comprises a TRPV1 antagonist and sage extract. In at least one embodiment, the aqueous topical formulation comprises a TRPV1 antagonist and clary sage extract. In at least one embodiment, the aqueous topical formulation comprises a TRPV1 antagonist and Oryza sativa extract. In at least one embodiment, the aqueous topical formulation comprises a TRPV1 antagonist and Zingiber officinale extract. In at least one embodiment, the aqueous topical formulation comprises a TRPV1 antagonist and ginger root extract. In at least one embodiment, the aqueous topical formulation comprises a TRPV1 antagonist and licorice extract. In at least one embodiment, the aqueous topical formulation comprises a TRPV1 antagonist and Matricaria recutita extract. In at least one embodiment, the aqueous topical formulation comprises a TRPV1 antagonist and oil palm extract.

In at least one embodiment, the aqueous topical formulation comprises a TRPV1 antagonist and tapioca extract. In at least one embodiment, the aqueous topical formulation comprises a TRPV1 antagonist and Vitis vinifera extract. In at least one embodiment, the aqueous topical formulation comprises a TRPV1 antagonist and grape extract. In at least one embodiment, the aqueous topical formulation comprises a TRPV1 antagonist and Corylus avellana extract. In at least one embodiment, the aqueous topical formulation comprises a TRPV1 antagonist and hazelnut extract. In at least one embodiment, the aqueous topical formulation comprises a TRPV1 antagonist and Turnera diffusa extract. In at least one embodiment, the aqueous topical formulation comprises a TRPV1 antagonist and Viola sororia extract. In at least one embodiment, the aqueous topical formulation comprises a TRPV1 antagonist and violet extract. In at least one embodiment, the aqueous topical formulation comprises a TRPV1 antagonist and Podocarpus totara extract. In at least one embodiment, the aqueous topical formulation comprises a TRPV1 antagonist and raspberry extract. In at least one embodiment, the aqueous topical formulation comprises a TRPV1 antagonist and Rubus idaeus extract. In at least one embodiment, the aqueous topical formulation comprises a TRPV1 antagonist and Rubus strigosus extract. In at least one embodiment, the aqueous topical formulation comprises a TRPV1 antagonist and wild indigo extract. In at least one embodiment, the aqueous topical formulation comprises a TRPV1 antagonist and Baptisia australis extract. In at least one embodiment, the aqueous topical formulation comprises a TRPV1 antagonist and Carthamus tinctorius extract. In at least one embodiment, the aqueous topical formulation comprises a TRPV1 antagonist and safflower extract. In at least one embodiment, the aqueous topical formulation comprises a TRPV1 antagonist and apple tree extract. In at least one embodiment, the aqueous topical formulation comprises a TRPV1 antagonist and apple extract.

In at least one embodiment, the aqueous topical formulation comprises a TRPV1 antagonist and Prunus mandschurica extract. In at least one embodiment, the aqueous topical formulation comprises a TRPV1 antagonist and apple tree leaf extract. In at least one embodiment, the aqueous topical formulation comprises a TRPV1 antagonist and cinnamon bark extract. In at least one embodiment, the aqueous topical formulation comprises a TRPV1 antagonist and strawberry extract. In at least one embodiment, the aqueous topical formulation comprises a TRPV1 antagonist and Angelica sinensis extract. In at least one embodiment, the aqueous topical formulation comprises a TRPV1 antagonist and acai oil extract.

In at least one embodiment, the aqueous topical formulation comprises a TRPV1 antagonist, phenyethyl alcohol, and an extract of Akebia quinata. In at least one embodiment, the aqueous topical formulation comprises a TRPV1 antagonist, phenyethyl alcohol, coconut liquid endosperm, coconut water, coconut fruit juice, and Rosa damascena extract. In at least one embodiment, the aqueous topical formulation comprises a TRPV1 antagonist, phenyethyl alcohol, and Cucumis sativus fruit extract.

The aqueous topical formulations disclosed herein can further comprise a stabilizer, emulsifier, additive, and/or preservative such as sodium benzoate, potassium sorbate, xanthan gum, a lecithin or a modified lecithin such as hydrogenated lecithin, gluconolactone, hydroxypropyl starch or a modified starch such as hydroxypropyl starch phosphate, a styrene/acrylate copolymer, phytic acid or a salt thereof, sodium laurel sulfoacetate, or maltodextrin. In at least one embodiment, the aqueous topical formulation comprises a TRPV1 antagonist and sodium benzoate. In at least one embodiment, the aqueous topical formulation comprises a TRPV1 antagonist and potassium sorbate. In at least one embodiment, the aqueous topical formulation comprises a TRPV1 antagonist and xanthan gum. In at least one embodiment, the aqueous topical formulation comprises a TRPV1 antagonist and a lecithin. In at least one embodiment, the aqueous topical formulation comprises a TRPV1 antagonist and hydrogenated lecithin. In at least one embodiment, the aqueous topical formulation comprises a TRPV1 antagonist and gluconolactone. In at least one embodiment, the aqueous topical formulation comprises a TRPV1 antagonist and hydroxypropyl starch. In at least one embodiment, the aqueous topical formulation comprises a TRPV1 antagonist and a styrene/acrylate copolymer. In at least one embodiment, the aqueous topical formulation comprises a TRPV1 antagonist and sodium phytate. In at least one embodiment, the aqueous topical formulation comprises a TRPV1 antagonist and phytic acid or a salt thereof. In at least one embodiment, the aqueous topical formulation comprises a TRPV1 antagonist and hydroxypropyl starch phosphate. In at least one embodiment, the aqueous topical formulation comprises a TRPV1 antagonist and maltodextrin.

In at least one embodiment, the aqueous topical formulation comprises a TRPV1 antagonist and at least one of sodium benzoate, potassium sorbate, xanthan gum, lecithin, hydrogenated lecithin, gluconolactone, hydroxypropyl starch, hydroxypropyl starch phosphate, a styrene/acrylate copolymer, phytic acid or a salt thereof, sodium laurel sulfoacetate, or maltodextrin. In at least one embodiment, the aqueous topical formulation comprises a TRPV1 antagonist, sodium benzoate, potassium sorbate, xanthan gum, hydrogenated lecithin, and maltodextrin. In at least one embodiment, the aqueous topical formulation comprises a TRPV1 antagonist, sodium benzoate, hydrogenated lecithin, gluconolactone, hydroxypropyl starch phosphate, a styrene/acrylate copolymer, sodium phytate, sodium laurel sulfoacetate, and maltodextrin. In at least one embodiment, the aqueous topical formulation comprises a TRPV1 antagonist, hydrogenated lecithin, and maltodextrin.

The total amount of plant extract in the aqueous topical formulations described herein, may be present in the formulation in an amount that is lower than the amount of the TRVP1 antagonist (w/w), it may be present in the formulation in an amount that is greater than the amount of the TRVP1 antagonist (w/w), or may be present in the formulation in an amount that is about the same as the amount of the TRVP1 antagonist (w/w).

In at least one embodiment, the aqueous topical formulation comprises water, pentapeptide-59, hydrogenated lecithin, shea butter, phenethyl alcohol, ethylhexylglycerin, maltodextrin, propylene glycol, xanthan gum, glycerin, Hamamelis virginiana leaf extract, Akebia quinata extract, sodium benzoate, potassium sorbate, sodium hyaluronate, coconut oil, tocopheryl acetate, and lactic acid. In at least one embodiment, the aqueous topical formulation comprises a TRPV1 antagonist, water, propylene glycol, xanthan gum, glycerin, Hamamelis virginiana leaf extract, Akebia quinata extract, sodium benzoate, potassium sorbate, sodium hyaluronate, coconut oil, tocopheryl acetate, and lactic acid.

In at least one embodiment, the aqueous topical formulation comprises water, sodium lauryl sulfoactetate, hydroxypropyl starch phosphate, pentylene glycol, glycerin, gluconolactone, sodium benzoate, styrene/acrylate copolymer, arginine, sodium phytate, Chamomilla recutita flower extract, coconut liquid endosperm, coconut water, coconut fruit juice, Rosa damascene flower extract, pentapeptide-59, hydrogenated lecithin, shea butter, phenethyl alcohol, ethylhexylglycerin, maltodextrin, Avena sativa oat kernel flour, tocopherol, and Sapindus trifoliatus fruit extract. In at least one embodiment, the aqueous topical formulation comprises a TVRP1 antagonist, water, sodium lauryl sulfoactetate, hydroxypropyl starch phosphate, pentylene glycol, glycerin, gluconolactone, sodium benzoate, styrene/acrylate copolymer, arginine, sodium phytate, Chamomilla recutita flower extract, coconut extract, Rosa damascene flower extract, Avena sativa oat kernel flour, tocopherol, and Sapindus trifoliatus fruit extract.

In at least one embodiment, the aqueous topical formulation comprises a water, pentylene glycol, palmitoyl-lysyl-valyl-lysine acetate salt, glycerin, sorbitan oleate decylglucoside crosspolymer, Aloe barbadensis leaf juice, pentapeptide-59, hydrogenated lecithin, shea butter, phenethyl alcohol, ethylhexylglycerin, maltodextrin, propylene glycol, menthoxypropanediol, betaine, hydrolyzed quinoa, Cucumis sativus fruit extract, and sodium hyaluronate. In at least one embodiment, the aqueous topical formulation comprises a TRPV1 antagonist, water, pentylene glycol, palmitoyl-lysyl-valyl-lysine acetate salt, glycerin, sorbitan oleate decylglucoside crosspolymer, Aloe barbadensis leaf juice, propylene glycol, menthoxypropanediol, betaine, hydrolyzed quinoa, Cucumis sativus fruit extract, and sodium hyaluronate.

The aqueous topical formulations disclosed herein may include at least one pH adjusting component to adjust and/or maintain the formulation at an appropriate pH. For example, lactic acid may be used to adjust the pH. As noted above, a healthy vulvovaginal environment is acidic, with a pH ranging from about 3.5 to about 5.5, such as from a pH of about 3.5 to about 4.5. A comparatively high pH, such as a pH above 4.5, can lead to, or be caused by, a change in the vaginal flora, such as by a Candida (yeast) infection and/or by reduced levels of estrogen.

In at least one embodiment, the aqueous topical formulation comprises at least one TRPV1 antagonist and at least one pH adjusting component. In at least one embodiment, the aqueous topical formulation comprises at least one TRPV1 antagonist and lactic acid. In at least one embodiment, the aqueous topical formulation comprises at least one TRPV1 antagonist and has a pH that is from about 3.0 to about 7.5. In at least one embodiment, the aqueous topical formulation comprises at least one TRPV1 antagonist and has a pH that is from about 3.5 to about 5.5. In at least one embodiment, the aqueous topical formulation comprises at least one TRPV1 antagonist and has a pH that is from about 3.5 to about 4.5. In at least one embodiment, the aqueous topical formulation comprises at least one TRPV1 antagonist and has a pH that is about 4. In at least one embodiment, the aqueous topical formulation comprises at least one TRPV1 antagonist and has a pH that is about 5. In at least one embodiment, the aqueous topical formulation comprises at least one TRPV1 antagonist and has a pH that is about 6. In at least one embodiment, the aqueous topical formulation comprises at least one TRPV1 antagonist and has a pH that is about 7.

The aqueous topical formulations disclosed herein may have an osmolality that is appropriate for a feminine intimate product. That is, the product may have an osmolality similar to that of, or iso-osmolar with, a normal vaginal secretion, including in a range from about 260 to about 295 mOsm/kg. A product with a comparatively high osmolality may draw water out of the vulvovaginal environment. In at least one embodiment, the aqueous topical formulation has an osmolality ranging from about 150 to about 400 mOsm/kg. In at least one embodiment, the aqueous topical formulation has an osmolality ranging from about 200 to about 400 mOsm/kg. In at least one embodiment, the aqueous topical formulation has an osmolality ranging from about 250 to about 350 mOsm/kg. In at least one embodiment, the aqueous topical formulation has an osmolality ranging from about 260 to about 280 mOsm/kg. In at least one embodiment, the aqueous topical formulation has an osmolality that is not greater than about 400 mOsm/kg, not greater than about 380 mOsm/kg, not greater than about 375 mOsm/kg, or not greater than about 350 mOsm/kg. In at least one embodiment, the aqueous topical formulation has an osmolality that is not less than about 150 mOsm/kg, not less than about 200 mOsm/kg, not less than about 300 mOsm/kg, not less than about 350 mOsm/kg, not less than about 380 mOsm/kg, or not less than about 400 mOsm/kg.

The aqueous topical formulations disclosed herein can be in any form that allows for topical exposure to the subject in a hypoestrogenic state and/or exhibiting menopausal symptoms, for example, emulsions, gels, foams, serums, or lotions for feminine intimate areas; bath milk; bath soak; body mist; body lotion; or serum. In at least one embodiment, the aqueous topical formulations disclosed herein is in the form of a bath milk or bath soak. In at least one embodiment, the aqueous topical formulations disclosed herein are in the form appropriate for feminine intimate areas, such as emulsions, gels, foams, serums, or lotions. In at least one embodiment, the aqueous topical formulations disclosed herein is in the form of a body mist. In at least one embodiment, the aqueous topical formulations disclosed herein is in the form of a lotion. In at least one embodiment, the aqueous topical formulations disclosed herein is in the form of a serum. In at least one embodiment, the aqueous topical formulations disclosed herein is in the form of at least one of a lotion, serum, bath milk, bath soak, or body mist.

The present disclosure also relates to method of treating at least one symptom of menopause in a subject suffering therefrom, comprising topically administering to the subject an effective amount of any of the aqueous topical formulations disclosed herein.

In some embodiments, the at least one symptom is related to vulvodynia. In some embodiments, the at least one symptom is related to vulvovaginal atrophy. In some embodiments, the at least one symptom is related to hot flashes. In some embodiments, the at least one symptom is related to night sweats. In some embodiments, the at least one symptom is related to dry skin as related to menopausal transition. In at least one embodiment, the at least one symptom is related to vulvodynia and/or vulvovaginal atrophy.

The present disclosure still further relates to the use of any of the aqueous topical formulations disclosed herein to help treat and/or ameliorate at least one symptom of menopause.

In at least one embodiment, the at least one symptom is chosen from vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

The present disclosure still further relates to the use of any of the aqueous topical formulations disclosed herein to help treat and/or ameliorate at least one symptom of reduced estrogen levels.

In at least one embodiment, the at least at least one symptom of reduced estrogen level is related to at least one of: premenopause, perimenopause, menopause, postmenopause, lactation, chemotherapy, radiation, an oophorectomy, an ovariotomy, a hysterectomy, administration of a selective estrogen receptor modulator, administration of a selective estrogen receptor degrader, administration of an antigonadotropin, a hypothalamic dysfunction, pregnancy failure, anorexia, polycystic ovarian syndrome, VVD, VVA, GSM, atrophic vaginitis, and vestibulodynia.

The formulation examples below illustrate the formulations according to the present disclosure without, however, limiting its scope.

### FORMULATION EXAMPLES

In the formulation examples described herein, the proportions of the various constituents are expressed as percentages by weight relative to the total weight of the composition.

### Example 1A. Intimate Serum Formulations

Formulations of a serum or lotion that is appropriate for topical application to the vulvovaginal area may include ranges of the components listed below:

| | Formulation ranges |
|---|---|
| Ingredient | (w/w) |
| Water | QSAD |
| Pentapeptide-59 (and) Hydrogenated Lecithin (and) | 0.0001 to 7, including |
| Butyrospermum Parkii (Shea) Butter (and) Phenethyl | 0.001-4, 0.01-3.5, 0.1-3, |
| Alcohol (and) Ethylhexylglycerin (and) Maltodextrin | 0.01-1.5, 0.1-1; 0.01-7, |
| (SensAmone P5) | 0.1-5, 0.5-3.5 |
| Propanediol | 0.001 to 20; including |
| | 0.01-10, 0.1-5, 0.5-4.5, 0.1- |
| | 3.5 |
| Xanthan Gum | 0.001 to 15, including |
| | 0.01-10, 0.1-5, 0.5-4.5, 0.1- |
| | 3.5 |
| Glycerin (and) Water (and) Akebia Quinata Extract | 0.001 to 10, including |
| | 0.01-8, 0.05-5, 0.1-3.5 |
| Sodium Benzoate | 0.001 to 10, including |
| | 0.01-8, 0.05-5, 0.1-3.5 |
| Potassium Sorbate | 0.001 to 10, including |
| | 0.01-8, 0.05-5, 0.1-3.5 |
| Glycerin (and) Hamamelis Virginiana (Witch Hazel) | 0.001 to 10, including |
| Leaf Extract | 0.01-8, 0.05-5, 0.1-3.5 |
| Sodium Hyaluronate | 0.00001 to 10, including |
| | 0.0001-5, 0.001-4, 0.01- |
| | 2.5, 0.1-1, 0.01-8, 0.05-5, |
| | 0.1-3.5 |
| Cocos Nucifera (Coconut) Oil | 0.0000001-1; 0.000001- |
| | 0.5, 0.00001-0.2, 0.0001- |
| | 0.1 |
| Tocopheryl Acetate (Vitamin E) | 0.0000001-2; 0.000001-1, |
| | 0.00001-0.1 |
| pH Adjuster | QS; such as 0.0001 to 10, |
| | including 0.001-1.0, 0.05- |
| | 0.75, 0.1-0.5, 0.5-2.0 |

An intimate serum of the following formulation was made:

| Ingredient | Formulation 1A |
|---|---|
| Water | QSAD |
| Pentapeptide-59 (and) Hydrogenated Lecithin | 2 |
| (and) Butyrospermum Parkii (Shea) Butter | |
| (and) Phenethyl Alcohol (and) Ethylhexylglycerin | |
| (and) Maltodextrin (SensAmone P5) | |
| Propanediol | 2 |
| Xanthan Gum | 1.3 |
| Glycerin (and) Water (and) Akebia Quinata Extract | 0.5 |
| Sodium Benzoate | 0.1 |
| Potassium Sorbate | 0.1 |
| Glycerin (and) Hamamelis Virginiana (Witch | 0.1 |
| Hazel) Leaf Extract | |
| Sodium Hyaluronate | 0.01 |
| Cocos Nucifera (Coconut) Oil | 0.000001 |
| Tocopheryl Acetate (Vitamin E) | 0.000001 |
| pH Adjuster | |
| Lactic Acid | 0.27 |

### Example 1B. User Feedback on Intimate Serums

A total of 31 women experiencing menopause were provided with an intimate serum formulation according to Formulation 1A. Of those women, a total of 27 participants used the intimate serum for the entire 4-week trial period. The participants were surveyed at the following intervals: pre-trial (27 participants were surveyed), 3-day (24), 1-week (24), 2-weeks (26), 3-weeks (28), and post-trial/4 weeks (27).

When asked after the trial period if the product provided “immediate hydration and moisturization,” 17 of 27 (63%) strongly agreed; 7 of 27 (26%) agreed; 2 of 27 (7%) neither agreed nor disagreed, and 1 of 27 (4%) disagreed. When asked after the trial period if the product “feels soothing upon application,” 18 of 27 (67%) strongly agreed; 8 of 27 (30%) agreed; zero neither agreed nor disagreed, and 1 of 27 (4%) disagreed. When asked after the trial period if the subject “felt a meaningful improvement in vaginal dryness,” 17 of 27 (63%) strongly agreed; 8 of 27 (30%) agreed; 2 of 27 (7%) neither agreed nor disagreed, and zero disagreed.

When asked before the trial began how they rated the severity of their vaginal dryness, on average, 5 of 27 (19%) rated their dryness as severe; 16 of 27 (60%) rated their dryness as moderate; 6 of 27 (22%) rated their dryness as mild, and no participant considered themself as having no vaginal dryness. Thus, 21 of 27 (78%) participants rated their dryness as severe or moderate before the trial period began. After a week of use, of the 24 participants that completed the survey, 18 of 24 (75%) participants rated the severity of their vaginal dryness as mild, with 2 of 24 (8%) rating as severe, 1 of 24 (4%) rating as moderate, and 3 of 24 (13%) rating as having no dryness. After 4 weeks of use, of the 27 participants that completed the survey, 9 of 27 (33%) participants rated the severity of their vaginal dryness as mild, with zero of 27 rating as severe, 3 of 27 (11%) rating as moderate, and 15 of 27 (56%) rating as having no dryness.

Before the trial began, a majority (24 of 27, or 67%) of women said their vaginal dryness was impacting their ability to enjoy a sexual relationship. After 4 weeks of use, 19 of 27 (70%) participants rated the improvement in their ability to fully enjoy their sexual relationship as significant or good, with 1 of 27 (7%) rating it as “some” improvement and 5 of 27 (19%) participants stating the question was not applicable.

### Example 2A. Bath Milk Formulations

Formulations of a bath milk or bath soak that is appropriate for topical application to the vulvovaginal area via the subject bathing or soaking in a bathtub, may include ranges of the components listed below: s

| | Formulation ranges |
|---|---|
| Formulation | (w/w) |
| Water | QSAD |
| Sodium Lauryl Sulfoacetate | 0.1 to 100; including |
| | 0.1-50, 0.5-25, 1-20 |
| Hydroxypropyl Starch Phosphate | 0.001 to 30; including |
| | 0.01-20, 0.1-10, 0.5-9.5, |
| | 1-8 |
| Pentapeptide-59 (and) Hydrogenated Lecithin (and) | 0.0001 to 7, including |
| Butyrospermum Parkii (Shea) Butter (and) Phenethyl | 0.001-4, 0.01-3.5, 0.1- |
| Alcohol (and) Ethylhexylglycerin (and) Maltodextrin | 3.0, 0.01-1.5, 0.1-1; |
| (Sensamone P5) | 0.01-7, 0.1-10, 0.1-5, |
| | 0.5-3.5 |
| Pentylene Glycol | 0.001 to 20; including |
| | 0.01-10, 0.1-5, 0.5-4.5, |
| | 0.1-3.5 |
| Glycerin | 0.001 to 15, including |
| | 0.01-10, 0.1-5, 0.5-4.5, |
| | 0.1-3.5 |
| Gluconolactone (and) Sodium Benzoate | 0.001 to 15, including |
| | 0.01-10, 0.1-5, 0.5-4.5, |
| | 0.1-3.5 |
| Styrene/Acrylates Copolymer (and) Aqua | 0.001 to 10, including |
| | 0.01-8, 0.05-5, 0.1-3.5 |
| Arginine | 0.00001 to 10, including |
| | 0.00001-3, 0.0001-2, |
| | 0.001-1.0, 0.01-0.5 |
| Sodium Phytate | 0.001 to 10, including |
| | 0.01-8, 0.05-5, 0.1-3.5 |
| Chamomilla Recutita (Matricaria) Flower Extract | 0.001 to 10, including |
| | 0.01-8, 0.05-5, 0.1-3.5 |
| Cocos Nucifera (Coconut) Liquid Endosperm/ | 0.0000001 to 10, |
| Cocos Nucifera (Coconut) Water (and) Glycerin | including 0.01-8, 0.05-5, |
| (and) Cocos Nucifera (Coconut) Fruit Juice | 0.1-3.5 |
| Rosa Damascena Flower Water | 0.0001 to 10, including |
| | 0.0001-5, 0.001-4, 0.01- |
| | 2.5, 0.1-1, 0.01-8, 0.05-5 |
| Avena Sativa (Oat) Kernel Flour | 0.0001 to 10, including |
| | 0.0001-5, 0.001-4, 0.01- |
| | 2.5, 0.1-1, 0.01-8, 0.05-5 |
| Tocopherol | 0.0000001-2; 0.000001- |
| | 1, 0.00001-0.1 |
| Sapindus Trifoliatus Fruit Extra | 0.0000001-2; 0.000001- |
| | 1, 0.00001-0.1 |
| pH adjuster | QS; such as 0.0001 to |
| | 10, including 0.001-1.0, |
| | 0.05-0.75, 0.1-0.5, 0.5- |
| | 2.0 |

Soothing bath milks of the following formulations were made:

| | Formulation | Formulation |
|---|---|---|
| Ingredient | 2A | 2B |
| Water | QSAD | QSAD |
| Sodium Lauryl Sulfoacetate | 16 | 16 |
| Hydroxypropyl Starch Phosphate | 7 | 7 |
| Pentapeptide-59 (and) Hydrogenated Lecithin (and) | 0.2 | 2 |
| Butyrospermum Parkii (Shea) Butter (and) Phenethyl | | |
| Alcohol (and) Ethylhexylglycerin (and) Maltodextrin | | |
| (Sensamone P5) | | |
| Pentylene Glycol | 2 | 2 |
| Glycerin | 1 | 1 |
| Gluconolactone (and) Sodium Benzoate | 1 | 1 |
| Styrene/Acrylates Copolymer (and) Aqua | 0.5 | 0.5 |
| Arginine | 0.5 | 0.5 |
| Sodium Phytate | 0.2 | 0.2 |
| Chamomilla Recutita (Matricaria) Flower Extract | 0.1 | 0.1 |
| Cocos Nucifera (Coconut) Liquid Endosperm/ | 0.1 | 0.1 |
| Cocos Nucifera (Coconut) Water (and) Glycerin | | |
| (and) Cocos Nucifera (Coconut) Fruit Juice | | |
| Rosa Damascena Flower Water | 0.05 | 0.05 |
| Avena Sativa (Oat) Kernel Flour | 0.01 | 0.01 |
| Tocopherol | 0.01 | 0.01 |
| Sapindus Trifoliatus Fruit Extra | 0.001 | 0.001 |

### Example 2B. User Feedback on Bath Milk

A total of 22 women experiencing menopause were provided with were provided with a bath milk formulation according to Formulation 2B. Of those women, a total of 15 participants used the bath milk for the 7 day trial period. The participants were surveyed at the following intervals: pre-trial (15 participants were surveyed) and post-trial/7 days (15).

When asked after the trial period if the product “does not irritate my intimate area,” 10 of 15 (67%) strongly agreed; 4 of 15 (27%) agreed; zero neither agreed nor disagreed, and 1 of 15 (7%) strongly disagreed. When asked after the trial period if the product “is quick and easy to use,” 9 of 15 (60%) strongly agreed; 5 of 15 (33%) agreed; zero neither agreed nor disagreed, and 1 of 15 (7%) strongly disagreed. When asked after the trial period if the product “is simple to incorporated into your daily wellness routine,” 10 of 15 (67%) strongly agreed; 2 of 15 (13%) agreed; 2 of 15 (13%) neither agreed nor disagreed, and 1 of 15 (7%) strongly disagreed. When asked after the trial period if the product “leaves me feeling calm and relaxed,” 6 of 15 (40%) strongly agreed; 7 of 15 (47%) agreed; 2 of 15 (13%) neither agreed nor disagreed, and zero disagreed.

When asked before the trial began how they rated the severity of their vaginal dryness, on average, 2 of 15 (13%) rated their dryness as severe; 7 of 15 (47%) rated their dryness as moderate; 3 of 15 (20%) rated their dryness as mild, and 3 of 15 (20%) participants considered themselves as having no vaginal dryness. Thus, 9 of 15 (60%) participants rated their dryness as severe or moderate before the trial period began. After a week of use, zero participants rated their dryness as severe, 3 of 15 (20%) rated the severity of their vaginal dryness as moderate, 5 of 15 (33%) rated as mild, and 7 of 15 (47%) as having no dryness.

Before the trial began, 7 of 15 (47%) of participants rated the severity of their vaginal discomfort or irritation as severe (3 of 15; 20%) or moderate (4 of 15; 27%). After 7 days of use, 7 of 15 (47%) participants reported having no vaginal discomfort.

### Example 3A. Cooling Mist Formulations

Formulations of a cooling mist that is appropriate for topical application may include ranges of the components listed below:

| Ingredient | Formulation ranges (w/w) |
|---|---|
| Water | QSAD |
| Pentylene Glycol | 0.001 to 30; including 0.01- |
| | 20, 0.1-10, 0.5-9.5, 1-8 |
| Palmitoyl Tripeptide-5 (and) Glycerin (and) Aqua | 0.001 to 20; including 0.01- |
| (Syn ®-Coll) | 10, 0.1-5, 0.5-4.5, 0.1-3.5 |
| Sorbitan Oleate Decylglucoside Crosspolymer | 0.001 to 20; including 0.01- |
| | 10, 0.1-5, 0.5-4.5, 0.1-3.5 |
| Aloe Barbadensis Leaf Juice | 0.001 to 15, including 0.01- |
| | 10, 0.1-5, 0.5-4.5, 0.1-3.5 |
| Pentapeptide-59 (and) Hydrogenated Lecithin (and) | 0.0001 to 7, including 0.0001- |
| Butyrospermum Parkii (Shea) Butter (and) Phenethyl | 4, 0.0001-3.5, 0.001-2.5, |
| Alcohol (and) Ethylhexylglycerin (and) Maltodextrin | 0.01-1.5, 0.1-1; 0.01-7, 0.1- |
| (Sensamone P5) | 10, 0.1-5, 0.5-3.5 |
| Menthoxypropanediol | 0.001 to 20, including 0.01- |
| | 10, 0.1-5, 0.5-4.5, 0.1-3.5 |
| Propanediol | 0.001 to 15, including 0.01- |
| | 10, 0.1-5, 0.5-4.5, 0.1-3.5 |
| Betaine | 0.00001 to 10, including |
| | 0.00001-3, 0.0001-2, 0.001-1, |
| | 0.01-0.5 |
| Hydrolyzed Quinoa | 0.001 to 10, including 0.01-8, |
| | 0.05-5, 0.1-3.5 |
| Glycerin (and) Cucumis Sativus (Cucumber) Fruit | 0.001 to 10, including 0.01-8, |
| Extract | 0.05-5, 0.1-3.5 |
| Sodium Hyaluronate | 0.00001 to 10, including |
| | 0.0001-5, 0.001-4, 0.01-2.5, |
| | 0.1-1, 0.01-8, 0.05-5, 0.1-3.5 |
| pH adjuster | QS; such as 0.0001 to 10, |
| | including 0.001-1.0, 0.05- |
| | 0.75, 0.1-0.5, 0.5-2.0 |

A cooling mist of the following formulation was made:

| Ingredient | Formulation 3A |
|---|---|
| Water | QSAD |
| Pentylene Glycol | 5 |
| Palmitoyl Tripeptide-5 (and) Glycerin (and) Aqua | 2 |
| (Syn ®-Coll) | |
| Sorbitan Oleate Decylglucoside Crosspolymer | 2 |
| Aloe Barbadensis Leaf Juice | 1 |
| Pentapeptide-59 (and) Hydrogenated Lecithin | 1 |
| (and) Butyrospermum Parkii (Shea) Butter | |
| (and) Phenethyl Alcohol (and) Ethylhexylglycerin | |
| (and) Maltodextrin (Sensamone P5) | |
| Menthoxypropanediol | 1 |
| Propanediol | 1 |
| Betaine | 0.5 |
| Hydrolyzed Quinoa | 0.1 |
| Glycerin (and) Cucumis Sativus (Cucumber) | 0.1 |
| Fruit Extract | |
| Sodium Hyaluronate | 0.0001 |

### Example 3B. User Feedback on Cooling Mist

A total of 33 women experiencing menopause were provided with a cooling mist formulation according to Formulation 3A. A total of 30 participants used the body mist for the seven day trial period. The participants were surveyed at the following intervals: pre-trial (30 participants were surveyed) and post-trial/7 days (30).

When asked after the trial period if the product “feels soothing upon application,” 17 of 30 (57%) strongly agreed; 11 of 30 (37%) agreed; 1 of 30 (3%) neither agreed nor disagreed, and 1 of 30 (3%) disagreed. When asked after the trial period if the product “leaves my skin feeling cooled,” 18 of 30 (60%) strongly agreed; 10 of 30 (33%) agreed; 1 of 30 (3%) neither agreed nor disagreed, and 1 of 30 (3%) disagreed. When asked after the trial period if the product “helped me sleep,” 12 of 30 (40%) strongly agreed; 11 of 30 (37%) agreed; 6 of 30 (20%) neither agreed nor disagreed, and 1 of 30 (3%) disagreed. When asked after the trial period if the product “gave me relief from my hot flashes,” 12 of 30 (40%) strongly agreed; 14 of 30 (47%) agreed; 3 of 30 (10%) neither agreed nor disagreed, and 1 of 30 (3%) disagreed. When asked after the trial period if the product “relieved night sweats,” 13 of 30 (43%) strongly agreed; 12 of 30 (40%) agreed; 2 of 30 (7%) neither agreed nor disagreed, and 2 of 30 (7%) disagreed.

When asked before the trial began how they rated the severity of their hot flashes, on average, 3 of 30 (10%) rated their hot flashes as severe; 23 of 30 (77%) rated their hot flashes as moderate; 2 of 30 (7%) rated their hot flashes as mild, and 2 of 30 (7%) participants reported no hot flashes. Thus, 26 of 30 (87%) participants rated their hot flashes as severe or moderate before the trial period began. After a week of use, zero participants rated their hot flashes as severe, 14 of 30 (47%) rated the severity of their hot flashes as moderate, 13 of 30 (43%) rated as mild, and 3 of 30 (10%) participants reported no hot flashes.

When asked before the trial began how they rated the severity of their night sweats, on average, 8 of 30 (27%) rated their night sweats as severe; 15 of 30 (50%) rated their night sweats as moderate; 6 of 30 (20%) rated their night sweats as mild, and 1 of 30 (3%) participants reported no night sweats. Thus, 23 of 30 (77%) participants rated their night sweats as severe or moderate before the trial period began. After a week of use, zero participants rated their night sweats as severe, 12 of 30 (40%) rated the severity of their night sweats as moderate, 14 of 30 (47%) rated as mild, and 4 of 30 (13%) participants reported no night sweats. Thus, 18 of 30 (60%) participants rated their night sweats as mild or did not report night sweats after the trial period ended.

### Exemplary Embodiments

Embodiment 1. An aqueous topical formulation comprising: at least one TRPV1 antagonist; and at least one plant extract chosen from Chamomilla recutita flower extract, Sapindus trifoliatus fruit extract, aloe leaf juice, cucumber extract, hydrolyzed quinoa, Hamamelis virginiana leaf extract, oat kernel extract, oat kernel flour, coconut extract, Rosa damascena extract, microalgae extract, Akebia quinate extract, Rhodosorus marinus extract, Phaeodactylum tricornutum extract, conifer extract, Blumea balsamifera extract, Kaempferia galanga extract, coriander extract, Coriandrum sativum extract, Citrus sinensis extract, lavender extract, Lavandula angustifolia extract, bay laurel extract, sweet basil extract, Ocimum basilicum extract, Ocimum tenuiflorum extract, mint extract, Mentha piperita extract, Mentha spicata extract, Mentha haplocalyx extract, citronella oil extract, rose oil extract, palmarosa oil extract, geranium extract, Cymbopogon citratus extract, Corymbia citriodora extract, Prunus mandschurica extract, apple tree leaf extract, cinnamon bark extract, strawberry extract, Angelica sinensis extract, acai oil extract, Mangifera indica extract, Fumaria officinalis extract, Rumex japonicus extract, guaiacwood extract, Guaiaicum officinale extract, Guaiacum sanctum extract, lemongrass extract, citrus oil extract, citrus bergamia extract, vanilla planifolia extract, vanilla tahitensis extract, Vanilla pompona extract, Theobroma cacao extract, pomegranate extract, Myrciaria dubia extract, Houttuynia cordata extract, sea lettuce extract, Ulva compressa extract, Agathosma betulina extract, Mentha canadensis extract, Eclipta prostrate extract, calendula extract, Silybum marianum extract, Cydonia oblonga extract, Oenothera biennis extract, Lepidium meyenii extract, Ulmus rubra extract, Olea europaea extract, Acmella oleracea extract, Humulus lupulus extract, Nicotiana sylvestris extract, jasmine extract, Cananga odorata extract, quince extract, olive tree extract, Vaccinium oxycoccus extract, Vaccinium macrocarpon extract, prickly pear cactus extract, Gymnema sylvestre extract, purslane extract, Rosa penduline extract, Ceratonia siliqua extract, Prunus amygdalus extract, Prunus dulcis extract, Prunus armeniaca extract, Ginkgo biloba extract, avocado extract, Persea americana extract, Camellia sinensis extract, eucalyptus extract, Linum usitatissimum extract, Cyamopsis tetragonoloba extract, sea buckthorn extract, Helianthus annuus extract, Simmondsia chinensis extract, Limnanthes alba extract, Sesamum indicum extract, Azadirachta indica extract, Moringa oleifera extract, Cedrus atlantica extract, calophyllum extract, Calophyllum inophyllum extract, Calophyllum tacamahaca extract, Daucus carota extract, Daucus carota sativa extract, Euphorbia cerifera extract, candelilla extract, carnauba palm extract, Echinacea purpurea extract, Zea mays extract, Quercus infectoria extract, arrowroot extract, Eurocoma longifolia extract, Trigonella foenum-graecum extract, salvia extract, rosemary extract, sage extract, clary sage extract, Oryza sativa extract, Zingiber officinale extract, licorice extract, Matricaria recutita extract, oil palm extract, tapioca extract, Vitis vinifera extract, hazelnut tree extract, Turnera diffusa extract, violet extract, Podocarpus totara extract, raspberry extract, Rubus idaeus extract, Rubus strigosus extract, wild indigo extract, safflower extract, and apple extract.

Embodiment 2. The aqueous topical formulation of embodiment 1, wherein the at least one TRPV1 antagonist is present in an amount ranging from 0.0001% w/w to 7% w/w, such as from 0.001% w/w to 4% w/w, from 0.01% w/w to 3.5% w/w, and from 0.1% w/w to 3.0% w/w.

Embodiment 3. The aqueous topical formulation of embodiment 1 or 2, wherein the total amount of plant extract is present in an amount ranging from 0.00001% w/w to 4.0% w/w, such as from 0.0001% w/w to 3.5% w/w, from 0.001% w/w to 2.5% w/w, from 0.01% w/w to 1.5%, and from 0.1% to 1% w/w.

Embodiment 4. The aqueous topical formulation of any of the preceding embodiments, further comprising sodium hyaluronate.

Embodiment 5. The aqueous topical formulation of embodiment 4, wherein sodium hyaluronate is present in an amount ranging from 0.0001% w/w to 5% w/w, such as from 0.001% w/w to 4% w/w, from 0.01% w/w to 2.5% w/w, and from 0.1% w/w to 1% w/w.

Embodiment 6. The aqueous topical formulation of any of the preceding embodiments, wherein the topical formulation further comprises vitamin E.

Embodiment 7. The aqueous topical formulation of embodiment 6, wherein the vitamin E is present in an amount ranging from 0.0000001% w/w to 2.0% w/w, such as from 0.000001% w/w to 1% w/w, and from 0.00001% w/w to 0.1% w/w.

Embodiment 8. The aqueous topical formulation of any of the preceding embodiments, wherein the topical formulation further comprises coconut oil.

Embodiment 9. The aqueous topical formulation of embodiment 8, wherein the coconut oil is present in an amount ranging from 0.0000001% w/w to 1.0% w/w, such as from 0.000001% w/w to 0.5% w/w, from 0.00001% w/w to 0.2%, and from 0.0001% to 0.1% w/w.

Embodiment 10. The aqueous topical formulation of any of the preceding embodiments, wherein the topical formulation further comprises betaine.

Embodiment 11. The aqueous topical formulation of embodiment 10, wherein the betaine is present in an amount ranging from 0.00001% w/w to 3.0% w/w, such as from 0.0001% w/w to 2.0%, from 0.001% w/w to 1.0% w/w, and from 0.01% w/w to 0.5% w/w.

Embodiment 12. The aqueous topical formulation of any of the preceding embodiments, wherein the topical formulation further comprises arginine.

Embodiment 13. The aqueous topical formulation of embodiment 12, wherein the arginine is present in an amount ranging from 0.00001% w/w to 3.0% w/w, such as from 0.0001% w/w to 2.0%, from 0.001% w/w to 1.0% w/w, and from 0.01% w/w to 0.5% w/w.

Embodiment 14. The aqueous topical formulation of any of the preceding embodiments, wherein the topical formulation further comprises niacinamide.

Embodiment 15. The aqueous topical formulation of embodiment 14, wherein the niacinamide is present in an amount less than 3.0% w/w, such as less than 2.0% w/w, less than 1.0% w/w, less than 0.5% w/w, less than 0.1% w/w, less than 0.01% w/w, or less than 0.0001% w/w.

Embodiment 16. The aqueous topical formulation of any of the preceding embodiments, wherein the topical formulation does not comprise at least one of niacinamide, estrogen, progesterone, paraben, phthalate, sulfate, gluten, fragrance, soy, nut, mineral oil, and formaldehyde; and preferably does not comprise niacinamide.

Embodiment 17. The aqueous topical formulation of any of the preceding embodiments, wherein the topical formulation further comprises at least one pH adjuster.

Embodiment 18. The aqueous topical formulation of embodiment 17, wherein the at least one pH adjuster comprises lactic acid.

Embodiment 19. The aqueous topical formulation of any of the preceding embodiments, wherein the topical formulation has a pH ranging from about 3.0 to about 7.5.

Embodiment 20. The aqueous topical formulation of embodiment 19, wherein the topical formulation has a pH ranging from about 3.5 to about 5.5.

Embodiment 21. The aqueous topical formulation of embodiment 20, wherein the topical formulation has a pH ranging from about 3.5 to about 4.5.

Embodiment 22. The aqueous topical formulation of any of the preceding embodiments, wherein the topical formulation has an osmolality ranging from about 150 mOsm/kg to about 400 mOsm/kg.

Embodiment 23. The aqueous topical formulation of embodiment 22, wherein the topical formulation has an osmolality ranging from about 200 mOsm/kg to about 400 mOsm/kg.

Embodiment 24. The aqueous topical formulation of embodiment 23, wherein the topical formulation has an osmolality ranging from about 250 mOsm/kg to about 350 mOsm/kg.

Embodiment 25. The aqueous topical formulation of embodiment 24, wherein the topical formulation has an osmolality ranging from about 260 mOsm/kg to about 280 mOsm/kg.

Embodiment 26. The aqueous topical formulation of embodiment 22, wherein the topical formulation has an osmolality not greater than about 400 mOsm/kg.

Embodiment 27. The aqueous topical formulation of embodiment 26, wherein the topical formulation has an osmolality not greater than about 380 mOsm/kg.

Embodiment 28. The aqueous topical formulation of embodiment 26, wherein the topical formulation has an osmolality not greater than about 350 mOsm/kg.

Embodiment 29. The aqueous topical formulation of any of the preceding embodiments, wherein the at least one TRPV1 antagonist is chosen from: JYL-1421 [N-(4-tert-butylbenzyl)-N′-[3-fluoro-4-(methylsulfonylamino)benzyl]thiourea]; KJM429 [N-(4-tert-butylbenzyl)-N′-[4-(methylsulfonylamino)benzyl]thiourea]; A-425619 [1-isoquinolin-5-yl-3-(4-trifluoromethyl-benzyl)-urea]; BCTC [N-(4-tertiarybutylphenyl)-4-(3-chloropyridin-2-yl)tetrahydropyrazine-1 (2H)-carbox-amide]; JNJ-17203212 [4-(3-trifluoromethylpyridin-2-yl) piperazine-1-carboxylic acid (5-trifluoromethylpyridin-2-yl)amide]; SB-705498 [N-(2-bromophenyl)-N′—[((R)-1-(5-trifluoromethyl-2-pyridyl)pyrrolidin-3-yl)]urea]; SB-366791 [4′-Chloro-3-methoxycinnamanilide]; AMG-9810 [(E)-3-(4-t-butylphenyl)-N-(2,3-dihydrobenzo[b][1,4]dioxin-6-yl)acrylamide]; AMG-2674 [3-Amino-5-[[2-[(2-methoxyethyl)amino]-6-[4-(trifluoromethyl)phenyl]-4-pyrimidinyl]oxy]-2 (1H)-quinoxalinone]; Capsazepine; MK-2295 [6-((R)-4-(6-(4-fluorophenyl)-2-((R)-2-methylpyrrolidin-1-yl)pyrimidin-4-yl)-3-methylpiperazin-1-yl)-5-methylnicotinic acid]; Ruthenium red; RRRRWW-NH₂; Methoctramine; AG-489; AG-505; DD-161515 [N-[2-(2-(N-methylpyrrolidinyl)ethyl]glycyl]-[N-[2,4-dichlorophenethyl]glycyl]-N-(2,4-dichlorophenethyl)glycinamide]; DD-191515 [[N-[3-(N,N-diethylamino)propyl]glycyl]-[N-[2,4-dichlorophenethyl]glycyl]-N-(2,4-dichlorophenethyl)glycinamide]; A-784168 [1-[3-(trifluoromethyl)pyridin-2-yl]-N-[4-(trifluoromethylsulfonyl)phenyl]-1,2,3,6-tetrahydropyridine-4-carboxamide]; A-795614 [N-1H-indazol-4-yl-N′-[(1R)-5-piperidin-1-yl-2,3-dihydro-1H-inden-1-yl]urea]; AMG-0347 [(E)-N-(7-hydroxy-5,6,7,8-tetrahydronaphthalen-1-yl)-3-(2-(piperidin-1-yl)-6-(trifluoromethyl)pyridin-3-yl)acrylamide]; AMG-517 [N-(4-[6-(4-trifluoromethyl-phenyl)-pyrimidin-4-yloxy]-benzothiazol-2-yl)-acetamide I]; Pentapeptide-59; Mariliance; Sensityl; resiniferatoxin, SYMSITIVE 1609 [4-tertiary butyl cyclohexane], Apritone [2-[(2E)-3,7-dimethyl-2,6-octadien-1-yl]cyclopentanone]; (−)-bornyl acetate; hydroxycitronellal [(7-hydroxy-3,7-dimethyloctanal]; methyl N,N-dimethylanthranilate; 2-ethoxy-3-ethylpyrazine; L-piperitone; isobornyl isobutyrate; 4-acetoxy-2,5-dimethyl-3 (2H)-furanone; tripropylamine; dihydrojasmone [3-methyl-2-pentylcyclopent-2-en-1-one]; 1-methyl-2-pyrole carboxaldehyde; 3-octyl acetate; 2-methylbutyl isovalerate; jasminone [2-(trans-2-pentenyl)cyclopentanone]; piperonyl isobutyrate; phenoxyethyl propionate; vanillin propylene glycol acetate; octenyl cyclopentanone; butyl isobutyrate; guaiacwood oil; tetrahydro-4-methyl-2-(2-methyl-1-propenyl)-2H pyran; and 4-tert-butyl cyclohexanol.

Embodiment 30. The aqueous topical formulation of any of the preceding embodiments, wherein the at least one TRPV1 antagonist is pentapeptide-59.

Embodiment 31. The aqueous topical formulation of embodiment 30, wherein the pentapeptide-59 comprises a lipid-based carrier system.

Embodiment 32. The aqueous topical formulation of embodiment 31 wherein the lipid-based carrier system comprises at least one of lecithin, shea butter, phenethyl alcohol, ethylhexylglycerin, and maltodextrin.

Embodiment 33. The aqueous topical formulation of embodiment 32, wherein the lipid-based carrier system comprises shea butter.

Embodiment 34. The aqueous topical formulation of embodiment 32, wherein the lipid-based carrier system comprises hydrogenated lecithin.

Embodiment 35. The aqueous topical formulation of any of the preceding embodiments, wherein the formulation is in a form chosen from: emulsion, gel, foam, serum, or lotion for feminine intimate areas; bath milk; bath soak; body mist; or emulsion, foam, serum or lotion for the body.

Embodiment 36. The aqueous topical formulation of embodiment 35, wherein the formulation is a serum.

Embodiment 37. The aqueous topical formulation of embodiment 36, wherein the formulation is a serum appropriate for application to feminine intimate areas.

Embodiment 38. The aqueous topical formulation of embodiment 35, wherein the formulation is a bath milk.

Embodiment 39. The aqueous topical formulation of embodiment 35, wherein the formulation is a bath soak.

Embodiment 40. The aqueous topical formulation of embodiment 35, wherein the formulation is a body mist.

Embodiment 41. The aqueous topical formulation of embodiment 40, wherein the formulation is a body mist applied to a subject via a pump spray bottle.

Embodiment 42. A method of treating at least one symptom of menopause in a subject suffering therefrom, comprising topically administering to the subject an effective amount of a formulation of any one of the preceding embodiments.

Embodiment 43. The method according to embodiment 42, wherein the at least one symptom is related to vulvodynia.

Embodiment 44. The method according to embodiment 42, wherein the at least one symptom is related to vulvovaginal atrophy.

Embodiment 45. The method according to embodiment 42, wherein the at least one symptom is related to hot flashes.

Embodiment 46. The method according to embodiment 42, wherein the at least one symptom is related to night sweats.

Embodiment 47. The method according to embodiment 42, wherein the at least one symptom is related to dry skin as related to menopausal transition.

Embodiment 48. The method according to embodiment 42, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

Embodiment 49. A method of treating at least one symptom of a reduced estrogen level in a subject suffering therefrom, comprising topically administering to the subject an effective amount of a formulation of any one of embodiments 1-41.

Embodiment 50. The method according to embodiment 49, wherein the at least one symptom is related to at least one of: premenopause, perimenopause, menopause, postmenopause, lactation, chemotherapy, radiation, an oophorectomy, an ovariotomy, a hysterectomy, administration of a selective estrogen receptor modulator, administration of a selective estrogen receptor degrader, administration of an antigonadotropin, a hypothalamic dysfunction, pregnancy failure, anorexia, polycystic ovarian syndrome, VVD, VVA, GSM, atrophic vaginitis, and vestibulodynia.

Embodiment 51. The method according to embodiment 49, wherein the at least one symptom is related to vulvodynia.

Embodiment 52. The method according to embodiment 49, wherein the at least one symptom is related to vulvovaginal atrophy.

Embodiment 53. The method according to embodiment 49, wherein the at least one symptom is related to hot flashes.

Embodiment 54. The method according to embodiment 49, wherein the at least one symptom is related to night sweats.

Embodiment 55. The method according to embodiment 49, wherein the at least one symptom is related to dry skin as related to menopausal transition.

Embodiment 56. The method according to embodiment 49, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

Embodiment 57. A method of ameliorating at least one symptom of menopause in a subject suffering therefrom, comprising topically administering to the subject an effective amount of a formulation of any one of embodiments 1-41.

Embodiment 58. The method according to embodiment 57, wherein the at least one symptom is related to vulvodynia.

Embodiment 59. The method according to embodiment 57, wherein the at least one symptom is related to vulvovaginal atrophy.

Embodiment 60. The method according to embodiment 57, wherein the at least one symptom is related to hot flashes.

Embodiment 61. The method according to embodiment 57, wherein the at least one symptom is related to night sweats.

Embodiment 62. The method according to embodiment 57, wherein the at least one symptom is related to dry skin as related to menopausal transition.

Embodiment 63. The method according to embodiment 57, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

Embodiment 64. A method of ameliorating at least one symptom of a reduced estrogen level in a subject suffering therefrom, comprising topically administering to the subject an effective amount of a formulation of any one of embodiments 1-41.

Embodiment 65. The method according to embodiment 64, wherein the at least one symptom is related to at least one of: premenopause, perimenopause, menopause, postmenopause, lactation, chemotherapy, radiation, an oophorectomy, an ovariotomy, a hysterectomy, administration of a selective estrogen receptor modulator, administration of a selective estrogen receptor degrader, administration of an antigonadotropin, a hypothalamic dysfunction, pregnancy failure, anorexia, polycystic ovarian syndrome, VVD, VVA, GSM, atrophic vaginitis, and vestibulodynia.

Embodiment 66. The method according to embodiment 64, wherein the at least one symptom is related to vulvodynia.

Embodiment 67. The method according to embodiment 64, wherein the at least one symptom is related to vulvovaginal atrophy.

Embodiment 68. The method according to embodiment 64, wherein the at least one symptom is related to hot flashes.

Embodiment 69. The method according to embodiment 64, wherein the at least one symptom is related to night sweats.

Embodiment 70. The method according to embodiment 64, wherein the at least one symptom is related to dry skin as related to menopausal transition.

Embodiment 71. The method according to embodiment 64, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

Embodiment 72. An aqueous topical serum comprising water, pentapeptide-59, lecithin, shea butter, phenethyl alcohol, ethylhexylglycerin, maltodextrin, propylene glycol, xanthan gum, glycerin, Hamamelis virginiana leaf extract, Akebia quinata extract, sodium benzoate, potassium sorbate, sodium hyaluronate, coconut oil, tocopheryl acetate, and lactic acid.

Embodiment 73. An aqueous topical serum comprising at least one TRPV1 antagonist, water, propylene glycol, xanthan gum, glycerin, Hamamelis virginiana leaf extract, Akebia quinata extract, sodium benzoate, potassium sorbate, sodium hyaluronate, coconut oil, tocopheryl acetate, and lactic acid.

Embodiment 74. An aqueous topical serum comprising at least one TRPV1 antagonist, water, and at least one of Hamamelis virginiana leaf extract, Akebia quinata extract, and a coconut extract.

Embodiment 75. The aqueous topical serum of any one of embodiments 72-74, wherein the topical formulation has a pH ranging from about 3.5 to about 5.5.

Embodiment 76. The aqueous topical serum of embodiment 75, wherein the topical formulation has a pH ranging from about 3.5 to about 4.5.

Embodiment 77. The aqueous topical serum of any one of embodiments 72-76, wherein the topical formulation has an osmolality raning from about 200 mOsm/kg to about 400 mOsm/kg.

Embodiment 78. The aqueous topical serum of embodiment 77, wherein the topical formulation has an osmolality ranging from about 250 mOsm/kg to about 350 mOsm/kg.

Embodiment 79. The aqueous topical serum of embodiment 78, wherein the topical formulation has an osmolality ranging from about 260 mOsm/kg to about 280 mOsm/kg.

Embodiment 80. The aqueous topical serum of embodiment 77, wherein the topical formulation has an osmolality not less than about 250 mOsm/kg.

Embodiment 81. The aqueous topical serum of embodiment 77, wherein the topical formulation has an osmolality not less than about 380 mOsm/kg.

Embodiment 82. The aqueous topical serum of embodiment 77, wherein the topical formulation has an osmolality not less than about 400 mOsm/kg.

Embodiment 83. A method of treating a subject suffering from at least one symptom of menopause, comprising topically administering to the subject an effective amount of the aqueous topical serum of any one of embodiments 72-82.

Embodiment 84. A method of treating a subject suffering from at least one symptom of a reduced estrogen level, comprising topically administering to the subject an effective amount of the aqueous topical serum of any one of embodiments 72-82.

Embodiment 85. The method of embodiment 83 or 84, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

Embodiment 86. The method of embodiment 83 or 84, wherein the at least one symptom is related to vulvodynia.

Embodiment 87. The method of embodiment 83 or 84, wherein the at least one symptom is related to vulvovaginal atrophy.

Embodiment 88. The method of embodiment 83 or 84, wherein the at least one symptom is related to dysparieunia.

Embodiment 89. The method of embodiment 83 or 84, wherein the at least one symptom is related to vaginal and/or vulvar dryness.

Embodiment 90. The method of embodiment 83 or 84, wherein the at least one symptom is related to dry skin as related to menopausal transition.

Embodiment 91. An aqueous topical bath milk or bath soak comprising water, sodium lauryl sulfoactetate, hydroxypropyl starch phosphate, pentylene glycol, glycerin, gluconolactone, sodium benzoate, styrene/acrylate copolymer, arginine, sodium phytate, Chamomilla recutita flower extract, coconut liquid endosperm, coconut water, coconut fruit juice, Rosa damascene flower extract, pentapeptide-59, hydrogenated lecithin, shea butter, phenethyl alcohol, ethylhexylglycerin, maltodextrin, Avena sativa oat kernel flour, tocopherol, and Sapindus trifoliatus fruit extract.

Embodiment 92. An aqueous topical bath milk or bath soak comprising at least one TRPV1 antagonist, water, sodium lauryl sulfoactetate, hydroxypropyl starch phosphate, pentylene glycol, glycerin, gluconolactone, sodium benzoate, styrene/acrylate copolymer, arginine, sodium phytate, Chamomilla recutita flower extract, coconut extract, Rosa damascene flower extract, Avena sativa oat kernel flour, tocopherol, and Sapindus trifoliatus fruit extract.

Embodiment 93. An aqueous topical serum comprising at least one TRPV1 antagonist, water, and at least one of Chamomilla recutita flower extract, coconut extract, Rosa damascene flower extract, Avena sativa oat kernel flour, and Sapindus trifoliatus fruit extract.

Embodiment 94. A method of treating a subject suffering from at least one symptom of menopause, comprising topically administering to the subject an effective amount of the aqueous topical milk bath or bath soak of any one of embodiments 91-93.

Embodiment 95. A method of treating a subject suffering from at least one symptom of a reduced estrogen level, comprising topically administering to the subject an effective amount of the aqueous topical milk bath or bath soak of any one of embodiments 91-93.

Embodiment 96. The method of embodiment 95 or 95, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

Embodiment 97. The method of embodiment 94 or 95, wherein the at least one symptom is related to vulvodynia.

Embodiment 98. The method of embodiment 94 or 95, wherein the at least one symptom is related to vulvovaginal atrophy.

Embodiment 99. The method of embodiment 94 or 95, wherein the at least one symptom is related to vaginal and/or vulvar dryness.

Embodiment 100. The method of embodiment 94 or 95, wherein the at least one symptom is related to vaginal and/or vulvar inflammation or vaginal and/or vulvar irritation.

Embodiment 101. The method of embodiment 94 or 95, wherein the at least one symptom is related to dry skin as related to menopausal transition.

Embodiment 102. An aqueous topical body mist comprising water, pentylene glycol, palmitoyl-lysyl-valyl-lysine acetate salt, glycerin, sorbitan oleate decylglucoside crosspolymer, Aloe barbadensis leaf juice, pentapeptide-59, hydrogenated lecithin, shea butter, phenethyl alcohol, ethylhexylglycerin, maltodextrin, propylene glycol, menthoxypropanediol, betaine, hydrolyzed quinoa, Cucumis sativus fruit extract, and sodium hyaluronate.

Embodiment 103. An aqueous topical body mist comprising at least one TRPV1 antagonist, water, water, pentylene glycol, palmitoyl-lysyl-valyl-lysine acetate salt, glycerin, sorbitan oleate decylglucoside crosspolymer, Aloe barbadensis leaf juice, propylene glycol, menthoxypropanediol, betaine, hydrolyzed quinoa, Cucumis sativus fruit extract, and sodium hyaluronate.

Embodiment 104. An aqueous topical body mist comprising at least one TRPV1 antagonist, water, and at least one of Aloe barbadensis leaf juice, hydrolyzed quinoa, and Cucumis sativus fruit extract.

Embodiment 105. A method of treating a subject suffering from at least one symptom of menopause, comprising topically administering to the subject an effective amount of the aqueous topical body mist of any one of embodiments 102-104.

Embodiment 106. A method of treating a subject suffering from at least one symptom of a reduced estrogen level, comprising topically administering to the subject an effective amount of the aqueous topical body mist of any one of embodiments 102-104.

Embodiment 107. The method of embodiment 105 or 106, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, and hyperalgesia in the region of the vulval vestibulus.

Embodiment 108. The method of embodiment 105 or 106, wherein the at least one symptom is related to vulvodynia.

Embodiment 109. The method of embodiment 105 or 106, wherein the at least one symptom is related to vulvovaginal atrophy.

Embodiment 110. The method of embodiment 105 or 106, wherein the at least one symptom is related to hot flashes.

Embodiment 111. The method of embodiment 105 or 106, wherein the at least one symptom is related to night sweats.

### EQUIVALENTS

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1-111. (canceled)

112. An aqueous topical formulation for genital, vulvar, and vaginal skin, comprising:
at least one TRPV1 antagonist selected from:
N-(4-tert-butylbenzyl)-N′-[3-fluoro-4-(methylsulfonylamino)benzyl]thiourea,
N-(4-tert-butylbenzyl)-N′-[4-(methylsulfonylamino)benzyl]thiourea,
1-isoquinolin-5-yl-3-(4-trifluoromethyl-benzyl)-urea,
N-(4-tertiarybutylphenyl)-4-(3-chloropyridin-2-yl)tetrahydropyrazine-1 (2H)-carboxamide,
4-(3-trifluoromethylpyridin-2-yl) piperazine-1-carboxylic acid (5-trifluoromethylpyridin-2-yl)amide,
N-(2-bromophenyl)-N′—[((R)-1-(5-trifluoromethyl-2-pyridyl)pyrrolidin-3-yl)]urea,
4′-chloro-3-methoxycinnamanilide,
(E)-3-(4-t-butylphenyl)-N-(2,3-dihydrobenzo[b][1,4]dioxin-6-yl)acrylamide,
3-amino-5-[[2-[(2-methoxyethyl)amino]-6-[4-(trifluoromethyl)phenyl]-4-pyrimidinyl]oxy]-2 (1H)-quinoxalinone,
capsazepine,
6-((R)-4-(6-(4-fluorophenyl)-2-((R)-2-methylpyrrolidin-1-yl)pyrimidin-4-yl)-3-methylpiperazin-1-yl)-5-methylnicotinic acid,
Ruthenium red,
RRRRWW-NH₂,
methoctramine,
N-[2-(2-(N-methylpyrrolidinyl)ethyl]glycyl]-[N-[2,4-dichlorophenethyl]glycyl]-N-(2,4-dichlorophenethyl)glycinamide,
[N-[3-(N,N-diethylamino)propyl]glycyl]-[N-[2,4-dichlorophenethyl]glycyl]-N-(2,4-dichlorophenethyl)glycinamide,
1-[3-(trifluoromethyl)pyridine-2-yl]-N-[4-(trifluoromethylsulfonyl)phenyl]-1,2,3,6-tetrahydropyridine-4-carboxamide,
N-1H-indazol-4-yl-N′-[(1R)-5-piperidin-1-yl-2,3-dihydro-1H-inden-1-yl]urea,
(E)-N-(7-hydroxy-5,6,7,8-tetrahydronaphthalen-1-yl)-3-(2-(piperidin-1-yl)-6-(trifluoromethyl)pyridine-3-yl)acrylamide,
N-(4-[6-(4-trifluoromethyl-phenyl)-pyrimidin-4-yloxy]-benzothiazol-2-yl)-acetamide,
pentapeptide-59,
resiniferatoxin,
4-tertiary butyl cyclohexane,
[2-[(2E)-3,7-dimethyl-2,6-octadien-1-yl]cyclopentanone,
(−)-bornyl acetate,
hydroxycitronellal [(7-hydroxy-3,7-dimethyloctanal],
methyl N,N-dimethylanthranilate,
2-ethoxy-3-ethylpyrazine,
L-piperitone,
isobornyl isobutyrate,
4-acetoxy-2,5-dimethyl-3 (2H)-furanone,
tripropylamine,
dihydrojasmone [3-methyl-2-pentylcyclopent-2-en-1-one],
1-methyl-2-pyrole carboxaldehyde,
3-octyl acetate,
2-methylbutyl isovalerate,
jasminone [2-(trans-2-pentenyl)cyclopentanone],
piperonyl isobutyrate,
phenoxyethyl propionate,
vanillin propylene glycol acetate,
octenyl cyclopentanone,
butyl isobutyrate,
guaiacwood oil, and
tetrahydro-4-methyl-2-(2-methyl-1-propenyl)-2H pyran;
water;
at least one humectant chosen from sodium hyaluronate, a glycol, and glycerin; and
at least one plant extract chosen from Akebia quinata extract, Helianthus annuus extract, and coconut extract.

113. The aqueous topical formulation of claim 112, wherein the TVPR1 antagonist comprises pentapeptide-59.

114. The aqueous topical formulation of claim 113, wherein the pentapeptide-59 is encapsulated in a lipid-based carrier system.

115. The aqueous topical formulation of claim 113, wherein the pentapeptide-59 is present in an amount ranging from 0.0001% w/w to 7% w/w.

116. The aqueous topical formulation of claim 114, wherein the pentapeptide-59 is encapsulated in a lipid-based carrier system and the system is present in an amount ranging from 0.0001% w/w to 7% w/w.

117. The aqueous topical formulation of claim 112, wherein the at least one humectant is present in an amount ranging from 0.00001% w/w to 10% w/w.

118. The aqueous topical formulation of claim 112, wherein:
(a) if present in the formulation, the Akebia quinata extract is present in an amount ranging from 0.001% w/w to 10% w/w;
(b) if present in the formulation, the Helianthus annuus extract is present in an amount ranging from 0.001% w/w to 10% w/w; or
(c) if present in the formulation, the coconut extract is present in an amount ranging from 0.0000001% w/w to 1.0% w/w.

119. The aqueous topical formulation of claim 112, wherein the formulation does not comprise at least one of niacinamide, estrogen, progesterone, paraben, phthalate, sulfate, gluten, soy, nut, mineral oil, and formaldehyde.

120. A method of treating at least one symptom of hormone fluctuation in a subject suffering therefrom, wherein the method comprises topically administering to the subject an effective amount of a formulation comprising:
at least one TRPV1 antagonist;
at least one plant extract chosen from Akebia quinata extract, Helianthus annuus extract, and coconut extract;
at least one humectant chose from sodium hyaluronate, a glycol, and glycerin;
vitamin E or any form thereof; and
sodium benzoate.

121. The method of claim 120, wherein the at least one TRPV1 antagonist is chosen from:
N-(4-tert-butylbenzyl)-N′-[3-fluoro-4-(methylsulfonylamino)benzyl]thiourea,
N-(4-tert-butylbenzyl)-N′-[4-(methylsulfonylamino)benzyl]thiourea,
1-isoquinolin-5-yl-3-(4-trifluoromethyl-benzyl)-urea,
N-(4-tertiarybutylphenyl)-4-(3-chloropyridin-2-yl)tetrahydropyrazine-1 (2H)-carboxamide,
4-(3-trifluoromethylpyridin-2-yl) piperazine-1-carboxylic acid (5-trifluoromethylpyridin-2-yl)amide,
N-(2-bromophenyl)-N′—[((R)-1-(5-trifluoromethyl-2-pyridyl)pyrrolidin-3-yl)]urea,
4′-chloro-3-methoxycinnamanilide,
(E)-3-(4-t-butylphenyl)-N-(2,3-dihydrobenzo[b][1,4]dioxin-6-yl)acrylamide,
3-amino-5-[[2-[(2-methoxyethyl)amino]-6-[4-(trifluoromethyl)phenyl]-4-pyrimidinyl]oxy]-2 (1H)-quinoxalinone,
capsazepine,
6-((R)-4-(6-(4-fluorophenyl)-2-((R)-2-methylpyrrolidin-1-yl)pyrimidin-4-yl)-3-methylpiperazin-1-yl)-5-methylnicotinic acid,
Ruthenium red,
RRRRWW-NH₂,
methoctramine,
N-[2-(2-(N-methylpyrrolidinyl)ethyl]glycyl]-[N-[2,4-dichlorophenethyl]glycyl]-N-(2,4-dichlorophenethyl)glycinamide,
[N-[3-(N,N-diethylamino)propyl]glycyl]-[N-[2,4-dichlorophenethyl]glycyl]-N-(2,4-dichlorophenethyl)glycinamide,
1-[3-(trifluoromethyl)pyridine-2-yl]-N-[4-(trifluoromethylsulfonyl)phenyl]-1,2,3,6-tetrahydropyridine-4-carboxamide,
N-1H-indazol-4-yl-N′-[(1R)-5-piperidin-1-yl-2,3-dihydro-1H-inden-1-yl]urea,
(E)-N-(7-hydroxy-5,6,7,8-tetrahydronaphthalen-1-yl)-3-(2-(piperidin-1-yl)-6-(trifluoromethyl)pyridine-3-yl)acrylamide,
N-(4-[6-(4-trifluoromethyl-phenyl)-pyrimidin-4-yloxy]-benzothiazol-2-yl)-acetamide,
pentapeptide-59,
resiniferatoxin,
4-tertiary butyl cyclohexane,
[2-[(2E)-3,7-dimethyl-2,6-octadien-1-yl]cyclopentanone,
(−)-bornyl acetate,
hydroxycitronellal [(7-hydroxy-3,7-dimethyloctanal],
methyl N,N-dimethylanthranilate,
2-ethoxy-3-ethylpyrazine,
L-piperitone,
isobornyl isobutyrate,
4-acetoxy-2,5-dimethyl-3 (2H)-furanone,
tripropylamine,
dihydrojasmone [3-methyl-2-pentylcyclopent-2-en-1-one],
1-methyl-2-pyrole carboxaldehyde,
3-octyl acetate,
2-methylbutyl isovalerate,
jasminone [2-(trans-2-pentenyl)cyclopentanone],
piperonyl isobutyrate,
phenoxyethyl propionate,
vanillin propylene glycol acetate,
octenyl cyclopentanone,
butyl isobutyrate,
guaiacwood oil, and
tetrahydro-4-methyl-2-(2-methyl-1-propenyl)-2H pyran.

122. The method of claim 121, wherein the at least one TRPV1 antagonist comprises pentapeptide-59.

123. The method of claim 122, wherein the pentapeptide-59 is encapsulated in a lipid-based carrier system.

124. The method of claim 122, wherein the pentapeptide-59 is present in an amount ranging from 0.0001% w/w to 7% w/w.

125. The method of claim 123, wherein the pentapeptide-59 is encapsulated in a lipid-based carrier system and the system is present in an amount ranging from 0.0001% w/w to 7% w/w.

126. The method of claim 120, wherein the at least one symptom is related to at least one of: premenopause, perimenopause, menopause, postmenopause, lactation, chemotherapy, radiation, an oophorectomy, an ovariotomy, a hysterectomy, administration of a selective estrogen receptor modulator, administration of a selective estrogen receptor degrader, administration of an antigonadotropin, a hypothalamic dysfunction, pregnancy failure, anorexia, polycystic ovarian syndrome, GSM, atrophic vaginitis, vestibulodynia, vulvodynia, vulvovaginal atrophy, and dry genital skin as related to hormone fluctuation.

127. The method of claim 120, wherein the at least one symptom is selected from the following: vaginal and/or vulvar dryness, irritation, burning sensations, dyspareunia, pain, throbbing, itching, stinging; frequent yeast infections, feeling of pressure, yellow malodorous discharge, tenderness, urinary frequency, incontinence, and urgency, urinary tract infections (UTIs), difficulty in sexual arousal, vaginal bleeding from fragile atrophic skin, dryness of labia, thinning and/or inflammation of the vaginal walls, decreased lubrication, compromised skin barrier function, rawness, spotting, allodynia, hyperalgesia in the region of the vulval vestibulus, a symptom related to vulvodynia, a symptom related to vulvovaginal atrophy, a symptom related to dyspareunia, a symptom related to vaginal and/or vulvar dryness, and a symptom related to dry genital skin as related to hormone fluctuation.

128. The method of claim 120, wherein the at least one humectant is present in the formulation in an amount ranging from 0.00001% w/w to 10% w/w.

129. The method of claim 120, wherein:
(a) if present in the formulation, the Akebia quinata extract is present in an amount ranging from 0.001% w/w to 10% w/w;
(b) if present in the formulation, the Helianthus annuus extract is present in an amount ranging from 0.001% w/w to 10% w/w; or
(c) if present in the formulation, the coconut extract is present in an amount ranging from 0.0000001% w/w to 1.0% w/w.

130. The method of claim 120, wherein the formulation does not comprise at least one of niacinamide, estrogen, progesterone, paraben, phthalate, sulfate, gluten, soy, nut, mineral oil, and formaldehyde.

131. The method of claim 120, wherein the formulation is topically administered to genital, vulvar, or vaginal skin.
